(19) United States
(12) Patent Application Publication
(10) Pub. No.: US 2026/0194526 A1
(43) Pub. Date: Jul. 9, 2026

(54) METHOD AND TREATMENT

(71) Applicant: ImmuneSignatures Pty Ltd, Concord West, New South Wales (AU)

(72) Inventors: Barbara Fazekas De St Groth, Sydney, New South Wales (AU); Helen McGuire, Sydney, New South Wales (AU); Natalie Smith, Sydney, New South Wales (AU)

(21) Appl. No.: 19/132,908

(22) PCT Filed: Nov. 24, 2023
(86) PCT No.: PCT/AU2023/051206

§ 371 Date: May 27, 2025

(30) Foreign Application Priority Data
Nov. 25, 2022 (AU) 2022903578

(51) Int. Cl. G01N 33/575; A61K 39/395; A61K 45/06; C07K 16/28

(52) U.S. Cl. CPC G01N 33/57585; A61K 39/3955; A61K 45/06; C07K 16/2818; C07K 16/2827; G01N 2333/70503; G01N 2333/7051; G01N 2333/70535; G01N 2333/70539; G01N 2333/70589; G01N 2333/70596; G01N 2333/978

(57) ABSTRACT

The present invention relates to a method of treating cancer in a human subject who is non-responsive to treatment with an immune checkpoint inhibitor, to a method of reducing non-responsiveness of a subject to treatment with an immune checkpoint inhibitor, to a method of determining whether a subject will be non-responsive to treatment with an immune checkpoint inhibitor, and to compositions and kits for such uses.

### FIELD

The present invention relates to a method of treating cancer in a human subject who is non-responsive to treatment with an immune checkpoint inhibitor, to a method of reducing non-responsiveness of a subject to treatment with an immune checkpoint inhibitor, and to compositions and kits for such uses.

### BACKGROUND

Immune checkpoint therapy is a form of cancer treatment that involves modulating the patient's immune system to treat a cancer by administering an immune checkpoint inhibitor. Immune checkpoint inhibitors target checkpoint regulators that suppress the body's immune response to cancer cells, such as programmed cell death protein1 (PD-1) or its ligand PD-L1.

Immune checkpoint inhibitors that have been approved, and/or are in clinical trials, for treatment of cancer, include the anti-PD-1 antibodies nivolumab (for metastatic melanoma), and pembrolizumab (for treatment of metastatic melanoma, lymphoma, mesothelioma, and non-small cell lung cancer), and the anti-PD-L1 antibodies including avelumab (for urothelial carcinoma, Merkel cell carcinoma, renal cell carcinoma) and atezolizumab (for urothelial carcinoma, non-small cell lung cancer (NSCLC), triple-negative breast cancer (TNBC), small cell lung cancer (SCLC) and hepatocellular carcinoma (HCC)).

However, a significant problem with treatment of cancer with immune checkpoint inhibitors is the large number of primary non-responders. For example, among metastatic melanoma patients treated with anti-PD-1 antibodies, approximately 60-70% of patients do not respond to treatment.

WO 2019/051542 describes methods for identifying subjects that are likely to be responsive to treatment with immune checkpoint inhibitors. While such an approach identifies and facilitates the treatment of those responsive to checkpoint inhibitors, the treatment of cancer patients who are non-responsive to immune checkpoint inhibitors remains problematic.

What is needed are improved methods for treatment of cancer in patients who are non-responsive to immune checkpoint inhibitors.

### SUMMARY

The inventors have found that a subset of cells which have high levels of intracellular arginase 1 play a significant role in the lack of responsiveness to immune checkpoint inhibitors that targets the interaction between PD-1 and its ligand.

As described in the Examples, the inventors have found that non-responsiveness to immune checkpoint therapy that targets the interaction between PD-1 and its ligand is closely correlated with an elevated population of a specific subset of the cells that have high intracellular levels of arginase 1. The inventors therefore envisage that by inhibiting the activity of arginase 1 in the subset of cells having high intracellular levels of arginase 1, the non-responsiveness of the subject to treatment with the immune checkpoint inhibitor can be reversed, thereby allowing the subject to subsequently be treated effectively with the immune checkpoint inhibitor.

Accordingly, a first aspect provides a method of treating cancer in a subject that has been identified as being non-responsive to treatment with an immune checkpoint inhibitor, comprising:
(a) administering an effective amount of an agent which inhibits arginase 1 activity and/or targets cells expressing arginase 1, for sufficient time to render the subject responsive to the immune checkpoint inhibitor; and
(b) administering an effective amount of the immune checkpoint inhibitor, optionally in combination with further administration of the agent which inhibits arginase 1 activity and/or targets cells expressing arginase 1
wherein the subject has been identified as non-responsive to the immune checkpoint inhibitor by
   (i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain and intracellular arginase 1;
   (ii) determining the distribution of cells in a blood sample from the subject within the series of classes of the positive response control and the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control

A second aspect provides a method of treating cancer in a human subject, comprising:
(a) assessing whether the subject will be non-responsive to treatment with an immune checkpoint inhibitor comprising:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain and intracellular arginase 1;
   (ii) determining the distribution of cells in a blood sample from the subject within the series of classes of the positive response control and the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control; and
(b) if the subject is determined to be non-responsive to the treatment, administering an effective amount of an agent which inhibits arginase 1 activity and/or targets cells expressing arginase 1, for sufficient time to render the subject responsive to the immune checkpoint inhibitor; and
(c) administering an effective amount of the immune checkpoint inhibitor, optionally in combination with further administration of the agent which inhibits arginase 1 activity and/or targets cells expressing arginase 1.

A third aspect provides a method of increasing responsiveness to treatment with an immune checkpoint inhibitor in a subject suffering from cancer that is non-responsive to the treatment, comprising administering to the subject an effective amount of an agent which inhibits arginase 1 activity, or targets cells expressing arginase 1, for sufficient time to render the subject responsive to the immune checkpoint inhibitor, wherein the subject has been determined to be non-responsive to the immune checkpoint inhibitor by:
(a) assessing the likelihood that the subject will be non-responsive to treatment with an immune checkpoint inhibitor comprising:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder and the non-responder within a series of classes, wherein the distribution of cells is according to the expression by each cell of the sample of a group of biomarkers so that cells having the same expression of biomarkers of the group are classified in the same class, wherein the group of biomarkers comprises CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells of a sample from the subject within the series of classes of the positive response control and the negative response control, the sample in the form of a sample of cells from blood of the subject;
   (iii) comparing the distribution of the cells of the sample from the subject within the series of classes with the negative response control and/or the positive response control, wherein the subject is determined to have a likelihood of being non-responsive to the treatment if the distribution of cells of the sample within the series of classes from the subject is substantially the same as the distribution of cells within the series of classes represented by the negative response control and/or is not the same as the distribution of cells within the series of classes represented by the positive response control.

In one embodiment, the group of biomarkers comprises at least 13 markers selected from CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase

In one embodiment, the group of biomarkers comprises the markers CD45, CD45RO, CD3, CD4, CD8, CD14, CD16, CD127, CD25, CD56, CD196 (CCR6), CD197 (CCR7), and Integrin beta7.

In one embodiment, the group of biomarkers comprises at least 13 markers selected from CD45, CD45RO, CD3, CD4, CD8, CD19, CD20, CD14, CD15, CD16, CD11b, CD66b, CD304, CD123, CD127, CD25, CD33, CD56, HLA-DR, CD38, CD192 (CCR2), CD196 (CCR6), CD197 (CCR7), Integrin beta7, GM-CSFR alpha chain, and intracellular arginase 1.

In one embodiment, the group of biomarkers comprises at least 13 markers selected from CD45, CD3, CD4, CD8, CD20, CD14, CD15, CD16, CD11b, CD66b, CD304, CD123, CD33, CD56, HLA-DR, CD38, CD192 (CCR2), GM-CSFR alpha chain, and intracellular arginase 1.

In one embodiment, the group of biomarkers comprises CD45, HLA-DR, CD11b, CD123, CD14, CD15, CD19, CD3, CD56 and intracellular arginase 1.

In one embodiment, the group of biomarkers comprises HLA-DR, CD14, CD16, CD66b, CD45RO, CD38 and intracellular arginase 1.

In one embodiment, the group of biomarkers comprises HLA-DR, CD3, CD14, CD16, CD19, CD66b, CD45RO, CD38 and intracellular arginase 1.

In one embodiment, the group of biomarkers comprises HLA-DR, CD14, CD16, CD66b, and intracellular arginase 1.

In one embodiment, the step (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
   thereby determining the distribution of cells of the sample from the subject within the series of classes of the response controls.

In one embodiment, the step (iii) comparing the distribution of the cells of the sample from the subject within the series of classes comprises the step of:
comparing the measured number of cells of the sample from the subject in each class of the series of classes of the response controls with the number of cells of the response controls in each class of the series of classes of the response controls;
thereby determining whether the distribution of the cells of the sample within the series of classes of the response controls is the same as the distribution of cells represented by one of the response control.

In one embodiment, the series of classes comprises naïve Tcon of CD4+, CD4+Treg of live, CD4+Tcon of live, beta7+ of CD45RO+CD4+Tcon, CCR6+ of CD45RO+CD4+Tcon, naïve CD8+ of live, naïve CD8+ of live, CD45RO+(of) CD8+, CD16loCD56 hi NK of live, CD14+CD16− classical monocytes of live, CD4+ of live, CD8+ of live, naïve of CD8+.

In one embodiment, the series of classes comprises the classes: CD8+ naïve % total, CD8+% total, CD8+CD45RO− naïve % total, B cells % total, CD3+% total, CD4+% total, CD4 naïve % total, CD4 naïve % CD4, CD4+TCM % total, Treg % total, Treg CD45RO+% total, IntB7% RO, CD8+CD45RO+ memory % CD8, NK cells % CD3-CD20−, CCR6% RO, Monocytes % total, CD14 monocytes % total, and NK cells % total. In one embodiment, the sample is a peripheral blood sample.

In one embodiment, the method further comprises monitoring the effectiveness of the treatment comprising the step of comparing the level of cells expressing intracellular arginase 1 in a blood sample from the subject after the treatment to the level of cells expressing intracellular arginase 1 before the treatment.

In one embodiment, the group of biomarkers comprises a CD4 or CD8 T cell marker, and/or a B cell or plasma cell marker and/or a NK cell marker and/or a monocyte or dendritic cell marker, and at least 13 markers selected from CD45, CD45RO, CD3, CD4, CD8, CD14, CD16, CD127, CD25, CD56, CD196 (CCR6), CD197 (CCR7), and Integrin beta7.

A fourth aspect provides a method of treating cancer in a subject that is non-responsive to treatment with an immune checkpoint inhibitor, comprising administering to the subject an effective amount of an agent which inhibits arginase 1 activity, or targets cells expressing arginase 1, for sufficient time to render the subject responsive to the immune checkpoint inhibitor, and administering an effective amount of the immune checkpoint inhibitor, optionally in combination with further administration of the agent which inhibits arginase 1 activity and/or targets cells expressing arginase 1.

A fifth aspect provides a method of treating cancer in a human subject, comprising:
(a) assessing whether the subject will be non-responsive to treatment with an immune checkpoint inhibitor, comprising determining the level of cells which express intracellular arginase 1 (ARG1+), and are CD14−, CD16−, CD66b−, in a blood sample from the subject relative to a positive response control, wherein the subject is determined to be non-responsive to the treatment if the level of cells in the sample which express intracellular arginase 1 (ARG1+) and are CD14−, CD16−, CD66b−, is elevated relative to a positive response control; and
(b) if the subject is determined to be non-responsive to the treatment, administering an effective amount of an agent which inhibits arginase 1 activity and/or targets cells expressing arginase 1, for sufficient time to render the subject responsive to the immune checkpoint inhibitor; and
(c) administering an effective amount of the immune checkpoint inhibitor, optionally in combination with further administration of the agent which inhibits arginase 1 activity and/or targets cells expressing arginase 1.

A sixth aspect provides a method of increasing responsiveness to treatment with an immune checkpoint inhibitor in a subject suffering from cancer that is non-responsive to the treatment, comprising administering to the subject an effective amount of an agent which inhibits arginase 1 activity, or targets cells expressing arginase 1, for sufficient time to render the subject responsive to the immune checkpoint inhibitor, wherein the subject has been determined to be non-responsive to the immune checkpoint inhibitor by
determining the level of cells which express intracellular arginase 1 (ARG1+) in a blood sample from the subject,
   wherein the subject is determined to be non-responsive to the treatment if the level of cells in the sample which express intracellular arginase 1 (ARG1+), and are CD14−, CD16−, CD66b−, is elevated relative to a positive response control.

In one embodiment, the cells which express intracellular arginase 1 (ARG1+) are CD14−, CD16−, and CD66b−.

In one embodiment, the cells which express intracellular arginase 1 (ARG1+) are CD14−, CD16−, CD66b−, and HLA-DR−.

In one embodiment, the cells which express intracellular arginase 1 (ARG1+) are CD14−, CD16−, and CD66b−, HLA-DR−, CD123+.

In one embodiment, the cells which express intracellular arginase 1 (ARG1+) are CD14−, CD16−, CD66b−, HLA-DR−, CD123+, CD45RO+, and CD38+.

In one embodiment, the cells which express intracellular arginase 1 (ARG1+) are CD14−, CD16−, CD66b−, HLA-DR−, CD45RO+, CD38+, CD19−, and CD3−.

In one embodiment, the cells which express intracellular arginase 1 (ARG1+) are CD3−, CD14−, CD16−, CD66b−, CD19−, HLA-DR−, CD45RO+ and CD38+.

In one embodiment of the fifth or sixth aspects, the positive response control is the level of cells which express intracellular arginase 1 (ARG1+), and are CD14−, CD16−, CD66b− in a blood sample from a subject or plurality of subjects that are responsive to treatment with the immune checkpoint inhibitor.

In one embodiment, the immune checkpoint inhibitor is an inhibitor of the interaction between PD-1 and its ligand.

In one embodiment, the immune checkpoint inhibitor is an anti-PD-1 antibody or anti-PD-L1 antibody.

In one embodiment, the immune checkpoint inhibitor is an anti-PD-1 monoclonal antibody, or an anti-PD-L1 monoclonal antibody.

In one embodiment, the immune checkpoint inhibitor is nivolumab, pembrolizumab, cemiplimab, durvalumab, atezolizumab or avelumab.

In one embodiment, the agent which targets cells expressing arginase 1 targets cells constitutively expressing arginase 1.

A seventh aspect provides a pharmaceutical composition comprising an agent which inhibits arginase 1 activity, or targets cells expressing arginase 1, and an immune checkpoint inhibitor.

An eighth aspect provides a kit for the treatment of cancer, comprising:
(a) an agent which inhibits arginase 1 activity, or targets cells expressing arginase 1; and optionally
(b) an immune checkpoint inhibitor.

A ninth aspect provides a method of assessing whether a subject suffering from cancer will be non-responsive to treatment with an immune checkpoint inhibitor, the method comprising:
(i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises intracellular arginase 1 and at least 4 markers selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, and GM-CSFR alpha chain;
(ii) determining the distribution of cells in a blood sample from the subject within the series of classes of the positive response control and the negative response control;
(iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

In one embodiment, the group of biomarkers comprises intracellular arginase 1 and at least 8 markers selected from CD45RO, CD3, CD4, CD8, CD19, CD20, CD14, CD15, CD16, CD11b, CD66b, CD304, CD123, CD127, CD25, CD33, CD56, HLA-DR, CD38, CD192 (CCR2), CD196 (CCR6), CD197 (CCR7), Integrin beta7, GM-CSFR alpha chain.

In one embodiment, the group of biomarkers comprises intracellular arginase 1 and at least 13 markers selected from CD45, CD3, CD20, CD14, CD15, CD16, CD11b, CD19, CD66b, CD304, CD123, CD33, CD56, HLA-DR, CD38, CD192 (CCR2), and GM-CSFR alpha chain.

In one embodiment, the group of biomarkers comprises intracellular arginase 1 and CD45, HLA-DR, CD11b, CD123, CD14, CD15, CD19, CD3, and CD56.

In one embodiment, the group of biomarkers comprises intracellular arginase 1 and CD3, HLA-DR, CD14, CD16, CD19, CD66b, CD45RO and CD38.

In one embodiment, the group of biomarkers comprises intracellular arginase 1 and CD3, CD45, HLA-DR, CD11b, CD123, CD14, CD15, CD19, and CD56.

In one embodiment, (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
thereby determining the distribution of cells of the sample from the subject within the series of classes of the response controls.

In one embodiment, (iii) comparing the distribution of the cells of the sample from the subject within the series of classes comprises the step of:
comparing the measured number of cells of the sample from the subject in each class of the series of classes of the response controls with the number of cells of the response controls in each class of the series of classes of the response controls;
thereby determining whether the distribution of the cells of the sample within the series of classes of the response controls is the same as the distribution of cells represented by one of the response control.

In one embodiment, the series of classes comprises the classes: CD8+ naïve % total, CD8+% total, CD8+CD45RO− naïve % total, B cells % total, CD3+% total, CD4+% total, CD4 naïve % total, CD4 naïve % CD4, CD4+TCM % total, Treg % total, Treg CD45RO+% total, IntB7% RO, CD8+CD45RO+ memory % CD8, NK cells % CD3-CD20−, CCR6% RO, Monocytes % total, CD14 monocytes % total, and NK cells % total.

A tenth aspect provides a method of determining whether a subject will be non-responsive to treatment with an immune checkpoint inhibitor, comprising determining the level of cells which are ARG1+, CD14−, CD16−, CD66b− in a blood sample from the subject; wherein the subject is determined to be non-responsive to the treatment if the level of cells in the sample which are ARG1+, CD14−, CD16−, CD66b− is elevated relative to a positive responder control.

In one embodiment, the ARG1+, CD14−, CD16−, CD66b− cells are ARG1+, CD14−, CD16− CD66b−, HLA-DR−.

In one embodiment, the ARG1+, CD14−, CD16−, CD66b− cells are ARG1+, CD14−, CD16− CD66b−, HLA-DR−, CD123+.

In one embodiment, the ARG1+, CD14−, CD16−, CD66b− cells are ARG1+, CD14−, CD16− CD66b−, HLA-DR−, CD123+, CD45RO+, CD38+.

In one embodiment, the ARG1+, CD14−, CD16−, CD66b− cells are ARG1+, CD14−, CD16−, CD66b−, HLA-DR−, CD45RO+, CD38+, CD19−, CD3−.

In one embodiment, the ARG1+, CD14−, CD16−, CD66b− cells are ARG1+, CD3−, CD14−, CD16−, CD66b−, CD19−, HLA-DR−, CD45RO+, CD38+.

In one embodiment, the immune checkpoint inhibitor is an inhibitor of the interaction between PD-1 and its ligand.

In one embodiment, the immune checkpoint inhibitor is an anti-PD-1 antibody or anti-PD-L1 antibody.

In one embodiment, the immune checkpoint inhibitor is an anti-PD-1 monoclonal antibody, or an anti-PD-L1 monoclonal antibody.

In one embodiment, the immune checkpoint inhibitor is nivolumab, pembrolizumab, cemiplimab, durvalumab, atezolizumab or avelumab.

In one embodiment, the blood sample is a peripheral blood sample.

In one embodiment, the cancer is non-small cell lung cancer (NSCLC) or colorectal cancer.

An alternative first aspect provides an agent which inhibits arginase 1 activity and/or targets cells expressing arginase 1 and an immune checkpoint inhibitor, for use in treating cancer in a subject that has been identified as being non-responsive to treatment with the immune checkpoint inhibitor;
or use of an agent which inhibits arginase 1 activity and/or targets cells expressing arginase 1, in the manufacture of a medicament for treating cancer in a subject that has been identified as being non-responsive to treatment with the immune checkpoint inhibitor, wherein the subject has been identified as non-responsive to the immune checkpoint inhibitor by
   (i) providing a positive response control from one or more positive responders who have formed a clinical response to the treatment, and/or a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and/or the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

An alternative second aspect provides an agent which inhibits arginase 1 activity and/or targets cells expressing arginase 1 and an immune checkpoint inhibitor, for use in treating cancer in a subject that has been identified as being non-responsive to treatment with the immune checkpoint inhibitor;
or use of an agent which inhibits arginase 1 activity and/or targets cells expressing arginase 1, in the manufacture of a medicament for treating cancer in a subject that has been identified as being non-responsive to treatment with the immune checkpoint inhibitor, wherein the subject has been identified as non-responsive to the immune checkpoint inhibitor by
   (i) providing a positive response control from one or more positive responders who have formed a clinical response to the treatment, and/or a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and/or the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

An alternative third aspect provides an agent which inhibits arginase 1 activity and/or targets cells expressing arginase 1, for use in increasing responsiveness to treatment with an immune checkpoint inhibitor in a subject suffering from cancer that is non-responsive to the treatment;
or use of an agent which inhibits arginase 1 activity and/or targets cells expressing arginase 1, in the manufacture of a medicament for increasing responsiveness to treatment with an immune checkpoint inhibitor in a subject suffering from cancer that is non-responsive to the treatment, wherein the subject has been identified as non-responsive to the immune checkpoint inhibitor by
   (i) providing a positive response control from one or more positive responders who have formed a clinical response to the treatment, and/or a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and/or the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

An alternative fourth aspect provides an agent which inhibits arginase 1 activity, or targets cells expressing arginase 1 and an immune checkpoint inhibitor, for use in treating cancer in a subject that is non-responsive to treatment with an immune checkpoint inhibitor; or use of an agent which inhibits arginase 1 activity, or targets cells expressing arginase 1 in the manufacture of a medicament for treating cancer in a subject in combination with an immune checkpoint inhibitor, wherein the subject is non-responsive to treatment with the immune checkpoint inhibitor.

An alternative fifth aspect provides an agent which inhibits arginase 1 activity, or targets cells expressing arginase 1, and an immune checkpoint inhibitor, for use in treating cancer in a subject that is non-responsive to treatment with an immune checkpoint inhibitor; or use of an agent which inhibits arginase 1 activity, or targets cells expressing arginase 1 in the manufacture of a medicament for treating cancer in a subject that is non-responsive to treatment with an immune checkpoint inhibitor in combination with an immune checkpoint inhibitor, wherein the subject has been identified as non-responsive to the immune checkpoint inhibitor by a method comprising:
(a) determining the level of cells which express intracellular arginase 1 (ARG1+), and are CD14−, CD16−, CD66b−, in a blood sample from the subject relative to a positive responder control,
wherein the subject is determined to be non-responsive to the treatment if the level of cells in the sample which express intracellular arginase 1 (ARG1+) and are CD14−, CD16−, CD66b−, is elevated relative to the positive responder control.

### BRIEF DESCRIPTION OF THE FIGURES

FIG. 1 is a heatmap showing the pre-therapy blood immune signature populations in 10 patients with NSCLC and a single healthy control. Data derived from cytometric analysis and population identification with a panel of 13 mAbs (specific for CD45, CD3, CD4, CD8, CD45RO, CD197 (CCR7), CD25, CD127, CD196 (CCR6), Integrin beta7, CD56, CD16, and CD14) were analysed in MeV. Columns show samples from each individual. Rows represent measured parameters (% cells in each subpopulation, row normalized to show SD from mean as indicated by the bar at top, with black indicating at least 1.5 SD below the mean and light grey indicating at least 2 SD above the mean). 5 patients failed to make a clinical response to anti-PD-L1 therapy (non-responder, indicated by the black bar). 5 patients made a clinical response to anti-PD-L1 therapy (responder, indicated by the green bar). Hierarchical clustering using Pearson correlation with average linkage indicated that data from the 5 non-responder patients clustered together, as did data from the 5 responder patients, illustrating the utility of the 13 mAb panel.

FIG. 2 shows dot-plots from mass cytometric analysis of a non-responder NSCLC patient. Arginase 1 expressing cells (0.37% of total) are gated (black polygons) and are clearly distinct from the remainder of cells expressing lineage markers (CD3, CD14, CD16, CD66b and CD19). Arginase 1 expressing cells are HLA-DR^{lo}, CD38^{hi} and CD45RO+.

FIG. 3 is a graph showing the percentage of intracellular arginase 1 expressing cells as a function of clinical response to anti-PD-1/PD-L1 therapy in NSCLC. **p<0.01

FIG. 4 is graphs showing lack of correlation between plasma arginase or arginine and clinical response to anti-PD-1/PD-L1 therapy in NSCLC. Left panel: Plasma arginase activity. Right panel: Plasma-arginine. ns not significant.

FIG. 5 is a graph showing correlation between plasma arginase activity and plasma arginine in NSCLC.

### DETAILED DESCRIPTION

A major problem with treatment of cancer with immune checkpoint inhibitors is the large number of subjects who do not respond to the treatment in the initial treatment period, such subjects referred to as primary non-responders. The inventors have previously found that in non-responders, the major cell populations that are decreased are CD4 T cells (specifically regulatory T cells (Tregs), naïve conventional (i.e., non-regulatory) CD4 T cells (here abbreviated as Tconv or Tcon), and central memory CD4 Tconv naïve CD8 T cells) and NK cells, with reciprocal increases in classical monocytes and HLA-DR⁺ cells (monocytes and B cells)). Without wishing to be bound by theory, the inventors believe that the decrease in naïve CD4 and CD8 T cells in non-responders is due to a systemic effect because naïve T cells circulate through the blood and secondary lymphoid tissues such as lymph nodes (LN) and spleen but are generally excluded from peripheral non-immune tissues such as tumours.

Naïve T cell numbers are regulated by thymic output and peripheral lifespan. As the age of cohorts tested by the inventors was older, and thymic output is low in older subjects, the inventors believe it is likely that reduced peripheral lifespan (i.e., reduced survival) was the likely immediate cause of the decrease in naïve CD4 and CD8 T cells. One of the most potent requirements for naïve T cell survival is low-level T cell receptor (TCR) signalling via dendritic cells in secondary lymphoid tissues. Peripheral T cells in which TCR expression is acutely abrogated die rapidly. Recognition of self-antigen in secondary lymphoid organs generates a partial phosphorylation response that prolongs naïve T cell survival.

The inventors believe that a decrease in TCR signalling would be predicted to reduce both T cell survival and T cell responsiveness to antigen. The inventors believe that reduced T cell responsiveness to antigen would account for the failure of tumour-primed cells to mount an effective attack against the tumour after administration of agents that interfere with the PD-1/PD-L1 pathway, while reduced T cell survival would account for the accompanying deficit in multiple T cell subsets.

A regulator of TCR signalling is the amino acid L-arginine. Plasma arginine concentrations are abnormally low in many cancer patients. Regulators of systemic L-arginine levels include the enzymes arginase 1 and nitric oxide synthase, encoded by the genes ARG1 and NOS2.

Recent immune studies in cancer have focused on the pre-therapy tumour microenvironment, where tumour-specific immune cells are vastly enriched compared to the circulation. In contrast, it has been reported that the T cells responsible for tumour clearance in response to anti-PD-1 therapy have a different TCR repertoire from the “exhausted” cells in the tumour microenvironment before checkpoint therapy. The inventors believe that the T cells responsible for tumour clearance in response to anti-PD-1 therapy must therefore have responded to anti-PD-1 somewhere other than in the tumour—the most likely site being the tumour draining lymph nodes—and then migrated into the tumour in response to therapy.

As described in the Examples, the inventors have analysed peripheral blood mononuclear cells from a cohort of non-small cell lung cancer patients prior to and after treatment with anti-PD-1/PD-L1 immune checkpoint inhibitor. The inventors have found a statistically significant negative correlation between the number of intracellular arginase 1-expressing cells in the blood and clinical response. The inventors have also found that correlations between clinical response and plasma arginase and arginine were not statistically significant (although as expected plasma arginine activity was higher in samples in which the arginine content was lower). This indicates that cells with high intracellular arginase 1 are a major driver of downregulation of T cell response, thereby compromising the T cell response to immune checkpoint inhibitors to the extent that an effective clinical response cannot be mounted.

The inventors therefore propose that the use of agents that target arginase 1 will increase the responsiveness of a subject suffering from cancer who is unresponsive to immune checkpoint inhibitors. Over time, this may revert the immune signature of non-response back to normal.

Accordingly, one aspect provides a method of treating cancer in a human subject, comprising:
(a) assessing whether the subject will be non-responsive to treatment with an immune checkpoint inhibitor comprising:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control; and
(b) if the subject is determined to be non-responsive to the treatment, administering an effective amount of an agent which inhibits arginase 1 activity and/or targets cells expressing arginase 1, for sufficient time to render the subject responsive to the immune checkpoint inhibitor; and
(c) administering an effective amount of the immune checkpoint inhibitor, optionally in combination with further administration of the agent which inhibits arginase 1 activity and/or targets cells expressing arginase 1.

In one aspect, there is provided a method of treating cancer in a human subject, comprising:
(a) assessing whether the subject will be non-responsive to treatment with an immune checkpoint inhibitor comprising:
   (i) providing a positive response control from one or more positive responders who have formed a clinical response to the treatment, and/or a negative response control from one or more non-responders who have not formed a clinical response to the treatment, each response control representing a distribution of cells of a blood sample from the positive responder(s) or the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and/or the negative response control;
   (iii) comparing the distribution of the cells of the sample from the subject within the series of classes with positive response control and/or the negative response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control; and
(b) if the subject is determined to be non-responsive to the treatment, administering an effective amount of an agent which inhibits arginase 1 activity and/or targets cells expressing arginase 1, for sufficient time to render the subject responsive to the immune checkpoint inhibitor; and
(c) administering an effective amount of the immune checkpoint inhibitor, optionally in combination with further administration of the agent which inhibits arginase 1 activity and/or targets cells expressing arginase 1.

Another aspect provides a method of treating cancer in a subject that has been identified as non-responsive to treatment with an immune checkpoint inhibitor, comprising:
(a) administering an effective amount of an agent which inhibits arginase 1 activity and/or targets cells expressing arginase 1, for sufficient time to render the subject responsive to the immune checkpoint inhibitor; and
(b) thereafter administering an effective amount of the immune checkpoint inhibitor, optionally in combination with further administration of the agent which inhibits arginase 1 activity and/or targets cells expressing arginase 1,
wherein the subject has been identified as non-responsive to the immune checkpoint inhibitor by:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

Another aspect provides a method of treating cancer in a subject that has been identified as non-responsive to treatment with an immune checkpoint inhibitor, comprising:
(a) administering an effective amount of an agent which inhibits arginase 1 activity and/or targets cells expressing arginase 1, for sufficient time to render the subject responsive to the immune checkpoint inhibitor; and
(b) thereafter administering an effective amount of the immune checkpoint inhibitor, optionally in combination with further administration of the agent which inhibits arginase 1 activity and/or targets cells expressing arginase 1,
wherein the subject has been identified as non-responsive to the immune checkpoint inhibitor by:
   (i) providing a positive response control from one or more positive responders who have formed a clinical response to the treatment, and/or a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells in a blood sample from the subject within the series of classes of the positive response control and/or the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

In one embodiment, the step (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
thereby determining the distribution of cells of the sample from the subject within the series of classes of the response controls.

In one embodiment, the step (iii) comparing the distribution of the cells of the sample from the subject within the series of classes comprises the step of:
comparing the measured number of cells of the sample from the subject in each class of the series of classes of the response controls with the number of cells of the response controls in each class of the series of classes of the response controls;
thereby determining whether the distribution of the cells of the sample within the series of classes of the response controls is the same as the distribution of cells represented by one of the response control.

### Types of Clinical Responses

A first step of the method comprises assessing whether a subject suffering from cancer of known responsiveness to treatment with an immune checkpoint inhibitor is likely to be response or non-responsive to the treatment with the immune checkpoint inhibitor.

A subject is responsive to the treatment with an immune checkpoint inhibitor if they exhibit a complete clinical response, a partial clinical response or stable disease.

A subject is non-responsive to the treatment with an immune checkpoint inhibitor if they exhibit progressive disease.

As generally understood, a “complete response” to therapy is generally understood as meaning the disappearance of all detectable signs of cancer in response to treatment. A “partial response” is generally understood as meaning a decrease in tumour load in an individual, for example in terms of tumour number, size and growth rate. A partial response may increase the time to disease progression. “Stable disease” is generally understood as meaning neither sufficient tumour shrinkage to qualify for partial response nor sufficient tumour increase to qualify for progressive disease.

A complete clinical response, a partial clinical response, stable or progressive disease may be determined in one embodiment by RECIST 1.0 criteria (Therasse P. et al.) 2000 J. Natl Cancer Inst 92:2015-16 as described below.

### RECIST 1.0 Criteria

### A. Definition of Measurable and Non-measurable Disease

Measurable disease: The presence of at least one measurable lesion.

Measurable lesion: Lesions that can be accurately measured in at least one dimension, with the longest diameter (LD) being:
≥20 mm with conventional techniques (medical photograph [skin or oral lesion], palpation, plain X-ray, CT, or MRI), OR
≥10 mm with spiral CT scan.

Non-measurable lesion: All other lesions including lesions too small to be considered measurable (longest diameter <20 mm with conventional techniques or <10 mm with spiral CT scan) including bone lesions, leptomeningeal disease, ascites, pleural or pericardial effusions, lymphangitis cutis/pulmonis, abdominal masses not confirmed and followed by imaging techniques, cystic lesions, or disease documented by indirect evidence only (e.g., by lab values).

### B. Methods of Measurement

Conventional CT and MRI: Minimum sized lesion should be twice the reconstruction interval. The minimum size of a baseline lesion may be 20 mm, provided the images are reconstructed contiguously at a minimum of 10 mm. MRI is preferred, and when used, lesions must be measured in the same anatomic plane by use of the same imaging sequences on subsequent examinations. Whenever possible, the same scanner should be used.

Spiral CT: Minimum size of a baseline lesion may be 10 mm, provided the images are reconstructed contiguously at 5 mm intervals. This specification applies to the tumors of the chest, abdomen, and pelvis.

Chest X-ray: Lesions on chest X-ray are acceptable as measurable lesions when they are clearly defined and surrounded by aerated lung. However, MRI is preferable.

Clinical Examination: Clinically detected lesions will only be considered measurable by RECIST criteria when they are superficial (e.g., skin nodules and palpable lymph nodes). In the case of skin lesions, documentation by color photography—including a ruler and patient study number in the field of view to estimate the size of the lesion—is required.

### C. Baseline Documentation of Target and Non-Target Lesions

All measurable lesions up to a maximum of five lesions per organ and ten lesions in total, representative of all involved organs, should be identified as target lesions and recorded and measured at baseline.

Target lesions should be selected on the basis of their size (lesions with the LD) and their suitability for accurate repeated measurements (either clinically or by imaging techniques). A sum of the LD for all target lesions will be calculated and reported as the baseline sum LD. The baseline sum LD will be used as a reference by which to characterize the objective tumor response.

All other lesions (or sites of disease) should be identified as non-target lesions and should also be recorded at baseline. Measurements of these lesions are not required, but the presence or absence of each should be noted throughout follow-up.

Documentation of indicator lesion(s) should include date of assessment, description of lesion site, dimensions, and type of diagnostic study used to follow lesion(s).

All measurements should be taken and recorded in metric notation, using a ruler or callipers.

### D. Response Criteria

Disease assessments are to be performed every 6 weeks after initiating treatment. However, subjects experiencing a partial or complete response must have a confirmatory disease assessment at least 28 days later. Assessment should be performed as close to 28 days later (as scheduling allows), but no earlier than 28 days.

Definitions for assessment of response for target lesion(s) are as follows:
Evaluation of Target Lesions
Complete Response (CR)—disappearance of all target lesions.
Partial Response (PR)—at least a 30% decrease in the sum of the LD of target lesions, taking as a reference, the baseline sum LD.
Stable Disease (SD)—neither sufficient shrinkage to qualify for PR nor sufficient increase to qualify for progressive disease (PD), taking as a reference, the smallest sum LD since the treatment started. Lesions, taking as a reference, the smallest sum LD recorded since the treatment started or the appearance of one or more new lesions.

### E. Evaluation of Non-Target Lesions

Definitions of the criteria used to determine the objective tumor response for non-target lesions are as follows:
Complete Response—the disappearance of all non-target lesions.
Incomplete Response/Stable Disease—the persistence of one or more non-target lesion(s).
Progressive Disease—the appearance of one or more new lesions and/or unequivocal progression of existing non-target lesions.

### F. Evaluation of Overall Response for RECIST-Based Response

The overall response is the best response recorded from the start of the treatment until disease progression/recurrence is documented. In general, the subject's best response assignment will depend on the achievement of both measurement and confirmation criteria.

The following table presents the evaluation of best overall response for all possible combinations of tumour responses in target and non-target lesions with or without the appearance of new lesions.

| Target Lesion | Non-Target Lesion | New Lesion | Overall response |
|---|---|---|---|
| CR | CR | No | CR |
| CR | Incomplete response/(SD) | No | PR |
| PR | Non-PD | No | PR |
| SD | Non-PD | No | SD |
| PD | Any | Yes or No | PD |
| Any | PD | Yes or No | PD |
| Any | Any | Yes | PD |

Note:
Subjects with a global deterioration of health status requiring discontinuation of treatment without objective evidence of disease progression at that time should be classified as having “symptomatic deterioration”. Every effort should be made to document the objective progression even after discontinuation of treatment.

In some circumstances, it may be difficult to distinguish residual disease from normal tissue. When the evaluation of complete response depends on this determination, it is recommended that the residual lesion be investigated (fine needle aspirate/biopsy) to confirm the complete response status.

### G. Confirmation Criteria

To be assigned a status of PR or CR, a confirmatory disease assessment should be performed no less than 28 days after the criteria for response are first met.

To be assigned a status of SD, follow-up measurements must have met the SD criteria at least once after study entry at a minimum interval of 12 weeks.

### Preparation of Response Controls

The method disclosed herein may include the further step of assessing one or more tissues or organs of an individual who has received the immune checkpoint inhibitor, to determine the regression of a tumour in the individual, and therefore to determine whether that individual is suitable for providing a positive or negative response control. In one embodiment the step utilises radiological imaging to determine the location and volume for each of the plurality of tumour lesions in the subject after treatment with the immune checkpoint inhibitor. For example, this can involve three-dimensional radiological images of the subject registering geographic locations of each of the plurality of tumour lesions. Non-limiting examples of radiological images that can be used to determine location and/or volume of a tumour lesion include positron emission tomography (PET) scans, x-ray computerized tomography (CT), magnetic resonance imaging (MRI), nuclear magnetic resonance imaging (NMRI), magnetic resonance tomography (MRT), or a combination thereof.

The positive and/or negative response control may be derived from a single individual. However, in some embodiments it is preferred that the positive response control is derived from a cohort or plurality of positive responders. In some embodiments, the negative response control is derived from a plurality of non-responders.

As described herein, where a cohort or plurality of positive responders or non-responder is used to derive a response control, the control is based on distribution data arising from an individual assessment of each of the positive responder or non-responder samples selected for derivation of the positive response control or negative response control. Thus each sample is assessed to determine a cell distribution profile specific to each sample. The cell distribution profiles of responders are then compiled to form the positive response control, and the cell distribution profiles of non-responders are compiled to form the negative response control.

The compilation may enable the identification of cell populations that are predominantly associated with lack of a clinical response to therapy in the majority of non-responders. The relevant statistical methods are understood by the skilled worker and described further herein.

In one embodiment, the positive response control describes the distribution of cells of a sample of a positive responder prior to therapy, or post therapy, of the positive responder with the immunomodulatory agent. Preferably the positive response control describes the distribution of cells of a positive responder, before the positive responder is subjected to treatment with the immunomodulatory agent leading to the positive response.

In one embodiment, the negative response control describes the distribution of cells of a sample of a non-responder prior to therapy, or post therapy, of the non-responder with the immune checkpoint inhibitor. Preferably the negative response control describes the distribution of cells of a non-responder, before the non-responder is subjected to treatment with the immune checkpoint inhibitor leading to the negative response.

As described herein, a purpose of the positive and negative response control is to provide a reference point against which a prediction of the likelihood of another individual not forming a clinical response to treatment with the same immune checkpoint inhibitor (i.e. same as used for formation of the positive and negative response controls) for, preferably, the same cancer (i.e. same as treated in the individual from which the positive and negative response control is derived) is made. The prediction is made on the basis of the comparison between test sample (sample from the subject) and positive and/or negative response controls. The comparison may be as between the frequency of cells in defined classes or subpopulations of cells of the positive and/or negative response control and the test sample.

In one embodiment, a response control may be identified according to the following steps:
(i) provide a responder cell sample in the form of cells of an individual who has responded to therapy with an immune checkpoint inhibitor
(ii) provide a non-responder cell sample in the form of cells of an individual who has not responded to therapy with an immune checkpoint inhibitor
(iii) apply a cell distribution analysis to the responder cell sample whereby the cells in the responder cell sample are distributed within a series of classes according to the expression by each cell of a group of biomarkers so that cells having the same expression of each biomarker of the group are classified in the same class, thereby forming a responder cell distribution profile
(iv) apply the same cell distribution analysis, albeit to the non-responder cell sample, thereby forming a non-responder cell distribution profile
(v) identify a distribution of cells in the responder cell distribution profile that is not seen in the non-responder cell distribution profile, and/or a distribution of cells in the non-responder cell distribution profile that is not seen in the responder cell distribution profile,
   wherein a distribution of cells in the responder cell distribution profile that is not seen in the non-responder cell distribution profile is identified as a positive response control, and/or a distribution of cells in the non-responder cell distribution profile that is not seen in the responder cell distribution profile is identified as a negative response control.

Typically, the responder cell distribution profile is a compilation of cell distribution profiles of different responders. This increases the likelihood that the control contains the cell distributions that are associated with response to therapy in the bulk of individuals who have responded or not responded to therapy.

Typically, the plurality of non responder cell samples may be a compilation of cell distribution profiles of different non responders.

The cells may be distributed according to the expression of at least 5, 6, 7, 8, 9, 10, 11, 12, or at least 13 biomarkers, although the number of biomarkers could be in some embodiments 20, 30, 40, 100 or more.

The phrase “same expression” in the context of “cells having the same expression”, and grammatical variants thereof will be generally understood as referring to a level of identity or similarity as between the relevant subjects. The level of identity or similarity may be a function of the number of responder cell distribution profiles that form the positive response control or the number of non-responder cell distribution profiles that form the negative response control. Where a response control arises from a compilation of responder or non-responder cell distribution profiles, it may be expected that “same expression” refers to a lower level of identity as between relevant subjects than might be expected where the response control is derived from a single responder or non-responder cell distribution profile.

### Biomarkers

In some embodiments the cells may be assessed for the absence of expression of a biomarker, or the presence of expression of a biomarker. In some embodiments the cells may be assessed for a particular level of expression of a biomarker. For example, cells may be expressed for a ‘low’ level of expression of a biomarker (for example, CD4^{lo}) or a ‘high’ level of expression of a biomarker (for example, CD4^{hi}). The meaning of level of biomarker expression referred to as ‘hi’ or ‘lo’ is generally well understood by the skilled worker. For example, the meaning of a CD4^{hi} and a CD4^{lo} T cell, or of a CD127^{hi} and a CD127^{lo} T cell is understood by the skilled worker and is routinely determined using standard techniques.

For the purpose of merely exemplifying the method, in one example, cells may be analysed to determine whether they can be distributed within the class CD25+CD127^{lo} T_{reg}. In this example, antibodies specific for CD25, CD127 and various other T_{reg} markers may be utilised. In this example, cells that have CD25⁺CD127^{lo} T_{reg} are distributed in this class. Cells that have CD25-CD127^{lo} T_{reg} or CD25⁺CD127^{hi}T_{reg} or CD25-CD127^{lo}T_{reg} are not distributed into the CD25⁺CD127^{lo} T_{reg} class.

Typically, the biomarkers are molecules that are characteristically expressed by cells having immune function, including lymphocytes, monocytes, granulocytes and the like. In one embodiment, the group of biomarkers includes a CD4 or CD8 T cell marker and at least 13 markers, typically at least 14, 15, 16, 17 or 18 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a CD4 or CD8 T cell marker and at least 19 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a CD4 or CD8 T cell marker and at least 29 markers, typically at least 30, 31, 32, 33, 34, 35, 36, 37, or 38 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10, and intracellular arginase 1.

In one embodiment, the group of biomarkers includes a CD4 or CD8 T cell marker and at least 39 markers from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes molecules that are expressed by B cells or plasma cells.

In one embodiment, the group of biomarkers includes a B cell or plasma cell marker and at least 13 markers, typically at least 14, 15, 16, 17 or 18 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a B cell or plasma cell marker and at least 19 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a CD4 or CD8 T cell marker and at least 29 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a B cell or plasma cell marker and at least 39 markers from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes molecules that are expressed by NK cells.

In one embodiment, the group of biomarkers includes a NK cell marker and at least 13 markers, typically at least 14, 15, 16, 17 or 18 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a NK cell marker and at least 19 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a NK cell marker and at least 29 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a NK cell marker and at least 39 markers from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes molecules that are expressed by monocytes or dendritic cells.

In one embodiment, the group of biomarkers includes a monocytes or dendritic cell marker and at least 13 markers, typically at least 14, 15, 16, 17 or 18 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a monocytes or dendritic cell marker and at least 19 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a monocyte or dendritic cell marker and at least 29 markers, typically at least 20, 21, 22, 23, 24, 25, 26, 27, or 28 markers, from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers includes a monocytes or dendritic cell marker and at least 39 markers from the group consisting of CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the group of biomarkers comprises, consists essentially of, or consists of, the markers CD45, CD45RO, CD3, CD4, CD8, CD14, CD16, CD127, CD25, CD56, CD196 (CCR6), CD197 (CCR7), and Integrin beta7.

In one embodiment, the group of biomarkers comprises, consists essentially of, or consists of, the markers CD45, CD45RO, CD3, CD4, CD8, CD14, CD16, CD127, CD25, CD56, CD196 (CCR6), CD197 (CCR7), and Integrin beta7. In one embodiment, using this group of biomarkers, the series of class may be: CD45RO+CD8+; CD14+, CD16−, classical monocytes of live; CCR6+ of CD45RO+, CD4+Tcon; CD4+ of live; CD4+Tcon of live; CD4+Treg of live; CD8+ of live; Beta7+ of CD45RO+CD4+Tcon; Naïve CD8+ of live; Naïve Tcon of CD4+; Naïve of CD8+; and CD16′° CD56^{hi}, NK of live.

In one embodiment, the group of biomarkers comprises, consists essentially of, or consists of, at least 13 markers selected from CD45, CD45RO, CD3, CD4, CD8, CD19, CD20, CD14, CD15, CD16, CD11b, CD66b, CD304, CD123, CD127, CD25, CD33, CD56, HLA-DR, CD38, CD192 (CCR2), CD196 (CCR6), CD197 (CCR7), Integrin beta7, GM-CSFR alpha chain, and intracellular arginase 1.

In one embodiment, the group of biomarkers comprises, consists essentially of, or consists of, intracellular arginase 1 and at least 4, 5, 6, 7, 8, 9, 10, 11 or at least 12 markers selected from CD45, CD45RO, CD3, CD4, CD8, CD19, CD20, CD14, CD15, CD16, CD11b, CD66b, CD304, CD123, CD127, CD25, CD33, CD56, HLA-DR, CD38, CD192 (CCR2), CD196 (CCR6), CD197 (CCR7), Integrin beta7, and GM-CSFR alpha chain.

In one embodiment, the group of biomarkers comprises, consists essentially of, or consists of, at least 13 markers selected from CD45, CD3, CD4, CD8, CD20, CD14, CD15, CD16, CD11b, CD66b, CD304, CD123, CD33, CD56, HLA-DR, CD38, CD192 (CCR2), GM-CSFR alpha chain, and intracellular arginase 1.

In one embodiment, the group of biomarkers comprises, consists essentially of, or consists of, intracellular arginase 1 and at least 4, 5, 6, 7, 8, 9, 10, 11, or at least 12 markers selected from CD45, CD3, CD4, CD8, CD20, CD14, CD15, CD16, CD11b, CD66b, CD304, CD123, CD33, CD56, HLA-DR, CD38, CD192 (CCR2), GM-CSFR alpha chain.

In one embodiment, the group of biomarkers comprises, consists essentially of, or consists of, CD45, HLA-DR, CD11b, CD123, CD14, CD15, CD19, CD3, CD56 and intracellular arginase 1.

In one embodiment, the group of biomarkers comprises, consists essentially of, or consists of, CD45, HLA-DR, CD11b, CD123, CD14, CD15, CD19, CD3, CD56 and intracellular arginase 1.

In one embodiment, the group of biomarkers comprises, consists essentially of, or consists of, HLA-DR, CD14, CD16, CD66b, CD45RO, CD38 and intracellular arginase 1.

In one embodiment, the group of biomarkers comprises, consists essentially of, or consists of, HLA-DR, CD3, CD14, CD16, CD19, CD66b, CD45RO, CD38 and intracellular arginase 1.

In one embodiment, the group of biomarkers comprises, consists essentially of, or consists of, HLA-DR, CD14, CD16, CD66b, and intracellular arginase 1.

In one embodiment, the positive response control may represent the distribution of cells within a series of at least 5, 10, 20, 30, 40 50, 100, 150, or 200 classes of cells.

In one embodiment, the negative response control may represent the distribution of cells within a series of at least 5, 10, 20, 30, 40 50, 100, 150, or 200 classes of cells.

In one embodiment, the cells of the test sample are not assessed for T cell or B cell receptor diversity.

The positive response control may describe the distribution of cells of a positive responder as the number of cells per class as a % of total number of cells. The negative response control may describe the distribution of cells of a non-responder as the number of cells per class as a % of total number of cells. In one embodiment, where the checkpoint inhibitor is an anti-PD-1 antibody, the distribution of cells that is assessed is at least 5, 6, 7, 8, 9 or at least 10 classes of cells according to Table 1 below.

In one embodiment, where the checkpoint inhibitor is an anti-PD-1 antibody, the distribution of cells that is assessed are ARG1+, and at least 4, 5, 6, 7, 8, or at least 9 classes of cells according to Table 1 below.

TABLE 1
Cell distribution

% CD3− CD56− CD14− of CD3−
% CD3− CD56− CD14− of live
% B cells of CD3− CD56− CD14−
% B cells of live
% Beta7+ of CD45RO+ Tcon CD4+
% Beta7+ of CD45RO+ Tregs
% CCR10+ of CD45RO+ Tcon CD4+
% CCR10+ of CD45RO+ Tregs
% CCR4+ of CD45RO+ Tcon CD4+
% CCR4+ of CD45RO+ Tregs
% CCR5+ of CD45RO+ Tcon CD4+
% CCR5+ of CD45RO+ Tregs
% CCR6+ of CD45RO+ Tcon CD4+
% CCR6+ of CD45RO+ Tregs
% CD11c+ mDCs of CD14− DCs
% CD11c+ mDCs of HLADR+ cells
% CD11c+ mDCs of live
% CD14− DCs of HLADR+ cells
% CD14− DCs of HLADR+ cells
% CD14− DCs of live
% CD14+ CD16− classical monocytes of HLADR+ cells
% CD14+ CD16− classical monocytes of live
% CD16+ CD14lo inflam monocytes of HLADR+ cells
% CD16+ CD14lo inflam monocytes of live
% CD16+ CD56lo NK cell of live
% CD16+ CD56lo of NK cell
% CD16lo CD56+ NK cell of live
% CD16lo CD56+ of NK cell
% CD20+ plasma cell of CD3− CD56− CD14−
% CD20+ plasma cell of live
% CD25+ CD127lo Tregs of CD4+
% CD25+ CD127lo Tregs of live
% CD25+ of FoxP3+ CD127lo Tregs
% CD25+ CD127lo Tregs of CD4+
% CD3− of live
% CD3− CD20− of live
% CD3+ of live
% CD304+ pDCs of CD14− DCs
% CD304+ pDCs of HLADR+ cells
% CD304+ pDCs of live
% CD4− CD8+ CD3+ of live
% CD4+ CD8− of CD3+
% CD4+ CD8− CD3+ of live
% CD4+ CD8+ of CD3+
% CD4+ CD8+ of live
% CD45RO− naive of Treg
% CD45RO− CCR7− effector CD8+ of live
% CD45RO− CCR7− effector of CD4+
% CD45RO− CCR7− effector of CD8+
% CD45RO− CCR7− effector of Tcon CD4+
% CD45RO− CCR7− effector Tcon CD4+ of live
% CD45RO− CCR7+ naive CD8+ of live
% CD45RO− CCR7+ naive of CD4+
% CD45RO− CCR7+ naive of CD8+
% CD45RO− CCR7+ naive of Tcon CD4+
% CD45RO− CCR7+ naive Tcon CD4+ of live
% CD45RO− naive CD8+ of live
% CD45RO− naive of CD8+
% CD45RO− naive of Tcon CD4+
% CD45RO− naive Tcon CD4+ of live
% CD45RO− naive Treg of live
% CD45RO+ memory of CD4+
% CD45RO+ memory of Tcon CD4+
% CD45RO+ memory of Treg
% CD45RO+ memory Tcon CD4+ of live
% CD45RO+ memory Treg of live
% CD45RO+ CCR7− memory of CD4+
% CD45RO+ CCR7− memory of Tcon CD4+
% CD45RO+ CCR7− memory Tcon CD4+ of live
% CD45RO+ CCR7− memory CD8+ of live
% CD45RO+ CCR7− memory of CD8+
% CD45RO+ CCR7+ central memory of CD4+
% CD45RO+ CCR7+ central memory of CD8+
% CD45RO+ CCR7+ central memory of Tcon CD4+
% CD45RO+ CCR7+ central memory Tcon CD4+ of live
% CD45RO+ CCR7+ central memory CD8+ of live
% CD45RO+ CCR7+ central memory of CD8+
% CD45RO+ memory CD8+ of live
% CD45RO+ memory of CD8+
% CD56+ NK cells of CD3− CD20−
% CD56+ NK cells of live
% CD8+ CD4− of CD3+
% CD86+ of inflam monocytes
% CD86+ of mDCs
% CD86+ of pDCs
% CLA+ of CD45RO+ Tcon CD4+
% CLA+ of CD45RO+ Tregs
% CXCR3+ of CD45RO+ Tcon CD4+
% CXCR3+ of CD45RO+ Tregs
% CXCR5+ of CD45RO+ Tcon CD4+
% CXCR5+ of CD45RO+ Tregs
% FcRL3+ of Tregs
% FoxP3+ CD25+ CD127lo Treg of live
% FoxP3+ of CD25+ CD127lo Tregs
% FoxP3+ CD127lo Tregs of CD4+
% FoxP3+ CD25+ CD127lo Tregs of CD4+
% GranzymeB+ of CD8+
% GranzymeB+ of NKT cell
% HLADR+ cells of CD3− CD20−
% HLADR+ cells of live
% live
% NKT cell of CD3+
% NKT cell of live
% OX40+ of CD45RO− CD8+
% OX40+ of CD45RO− Tcon CD4+
% OX40+ of CD45RO+ CD8+
% OX40+ of CD45RO+ Tcon CD4+
% OX40+ of NKT cell
% OX40+ of Tregs
% PD1+ of CD8+
% PD1+ of NKT cell
% PD1+ of Tcon CD4+
% PD1+ of Tregs
% PDL1+ of classic monocytes
% PDL1+ of inflam monocytes
% PDL1+ of mDCs
% PDL1+ of pDCs
% Tcon CD4+ of CD4+
% Tcon CD4+ of live
% Tigit+ of Tregs
% Tigit+ of NKT cell
% Tigit+ of CD8+
% GranzymeB+ of CD8+
% GranzymeB+ of NKT cell
% OX40+ of CD45RO+ Tcon CD4+
% OX40+ of CD45RO− Tcon CD4+
% OX40+ of Tregs
% OX40+ of CD45RO+ CD8+
% OX40+ of CD45RO− CD8+
% CD86+ of classic monocytes
% CD86+ of mDCs
% CD86+ of pDCs
% CD86+ of inflam monocytes
% FcRL3+ of Tregs
% PDL1+ of classic monocytes
% PDL1+ of mDCs
% PDL1+ of pDCs
% PDL1+ of inflam monocytes
% CCR4+ of CD45RO+ Tcon CD4+
% CCR5+ of CD45RO+ Tcon CD4+
% CCR6+ of CD45RO+ Tcon CD4+
% CCR10+ of CD45RO+ Tcon CD4+
% Beta7+ of CD45RO+ Tcon CD4+
% CLA+ of CD45RO+ Tcon CD4+
% CXCR3+ of CD45RO+ Tcon CD4+
% CCR5+ of CD45RO+ Tregs
% CCR6+ of CD45RO+ Tregs
% CCR10+ of CD45RO+ Tregs
% Beta7+ of CD45RO+ Tregs
% CLA+ of CD45RO+ Tregs
% CXCR3+ of CD45RO+ Tregs
% CXCR5+ of CD45RO+ Tregs

The phrase “substantially the same as the distribution” in the context of “determining whether the distribution of the cells of the test sample within the series of classes of the negative response control is the same as the distribution of cells represented by the negative response control” and grammatical variants thereof will be generally understood as referring to a level of similarity or identity between the test sample and the negative response control.

Preferably the distribution is the same across at least the majority of classes of the negative response control. In some embodiments, there may be some difference in distribution in a minority of classes of the negative response control.

In one embodiment, the cells of the responder and non responder are obtained from peripheral blood. The cells may be enriched for a leukocyte subset. For example, the cells may be enriched for mononuclear or polynuclear cells.

The assessment of the cells of a positive responder and/or non-responder who has been selected for formation of a response control may be performed by mass cytometry methods including TOF cytometry, according to the methodology described below for assessment of cells of the test sample, and as described in WO2019/051542.

Determining Distribution of Cells in Test Sample (i.e. The Sample from the Subject)

The distribution of cells in the test sample may be determined according to the following steps:
a. assessing each cell of the test sample for the expression of each biomarker of the group of biomarkers of the positive and/or negative response control;
b. classifying each cell of the test sample into one of the series of classes of the positive response control and/or the negative response control;
c. measuring the number of cells of the test sample in each class of the series of classes of the positive response control and/or the negative response control.

An assessment of the distribution of cells in the test sample is a consideration of the relative number of cells within each class of the series of classes of the positive and/or negative response control. This may also be expressed as the number of cells per class as a % of total number of cells of the test sample. These assessments may be completed by mass cytometry methods including TOF cytometry, which enables individual cell assessment for expression of a large number of biomarkers (40 or more) per cell See: [Spitzer M H and G P Nolan 2016 Cell 165: 780-791].

### Comparing Test Sample and Response Controls

The determination of whether the distribution of the cells of the test sample within the series of classes of the negative response control is the same as the distribution of cells represented by the negative response control generally requires a step of:
a. comparing the measured number of cells of the test sample in each class of the series of classes of the negative response control with the number of cells of the negative response control in each class of the series of classes of the negative response control. The determination of whether the distribution of the cells of the test sample within the series of classes of the positive response control is different from the distribution of cells represented by the positive response control generally requires a step of:
b. comparing the measured number of cells of the test sample in each class of the series of classes of the positive response control with the number of cells of the positive response control in each class of the series of classes of the positive response control. These steps can be performed in silico using available software packages.

In one embodiment, the cells which produce intracellular arginase 1 are myeloid cells. In one embodiment, the cells which produce intracellular arginase 1 are CD14−, CD16−.

In one embodiment, cells which produce intracellular arginase 1 express arginase 1 constitutively.

The term “determining” refers to “detecting” or “measuring” a level or a presence or absence of an item to be detected (for example, a cell type). As used herein, the term “level” refers to the amount of, number of, percentage of, or concentration of an item.

As mentioned above, the inventors have observed that T cells that respond to the immune checkpoint inhibitor are outside the tumour microenvironment. Accordingly, the sample is a sample from tissue outside of the tumour microenvironment. In this regard, the sample is a blood sample. In one embodiment, the blood sample is a peripheral blood sample. The sample may be a blood sample enriched for mononuclear or polynuclear cells.

If a subject is assessed or identified to be non-responsive to the immune checkpoint inhibitor treatment, the non-responsiveness can be at least partly reversed by administering to the subject an effective amount of an agent which inhibits arginase 1 activity and/or targets cells expressing arginase 1.

Another aspect provides a method of increasing responsiveness to treatment with an immune checkpoint inhibitor in a subject suffering from cancer that is non-responsive to treatment with the immune checkpoint inhibitor, the method comprising administering to the subject an effective amount of an agent which inhibits arginase 1 activity, or targets cells expressing arginase 1, for sufficient time to render the subject responsive to the immune checkpoint inhibitor.

Another aspect provides a method of increasing responsiveness to treatment with an immune checkpoint inhibitor in a subject suffering from cancer that is non-responsive to the treatment, comprising administering to the subject an effective amount of an agent which inhibits arginase 1 activity, or targets cells expressing arginase 1, for sufficient time to render the subject responsive to the immune checkpoint inhibitor, wherein the subject has been determined to be non-responsive to the immune checkpoint inhibitor by:
(a) assessing the likelihood that the subject will be non-responsive to treatment with an immune checkpoint inhibitor comprising:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder and the non-responder within a series of classes, wherein the distribution of cells is according to the expression by each cell of the sample of a group of biomarkers so that cells having the same expression of biomarkers of the group are classified in the same class, wherein the group of biomarkers comprises includes a CD4 or CD8 T cell marker, and/or a B cell or plasma cell marker and/or a NK cell marker and/or a monocyte or dendritic cell marker, and at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells of a sample from the subject within the series of classes of the positive response control and the negative response control, the sample in the form of a sample of cells from blood of the subject;
   (iii) comparing the distribution of the cells of the sample from the subject within the series of classes with the negative response control and/or the positive response control, wherein the subject is determined to have a likelihood of being non-responsive to the treatment if the distribution of cells of the sample within the series of classes from the subject is substantially the same as the distribution of cells within the series of classes represented by the negative response control and/or is not the same as the distribution of cells within the series of classes represented by the positive response control.

In one embodiment, the group of biomarkers comprises at least 13 markers selected from CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

Another aspect provides a method of increasing responsiveness to treatment with an immune checkpoint inhibitor in a subject suffering from cancer that is non-responsive to the treatment, comprising administering to the subject an effective amount of an agent which inhibits arginase 1 activity, or targets cells expressing arginase 1, for sufficient time to render the subject responsive to the immune checkpoint inhibitor, wherein the subject has been determined to be non-responsive to the immune checkpoint inhibitor by:
(a) assessing the likelihood that the subject will be non-responsive to treatment with an immune checkpoint inhibitor comprising:
   (i) providing a positive response control from one or more positive responders who have formed a clinical response to the treatment, and/or a negative response control from one or more non-responders who have not formed a clinical response to the treatment, each response control representing a distribution of cells of a blood sample from the positive responder and the non-responder within a series of classes, wherein the distribution of cells is according to the expression by each cell of the sample of a group of biomarkers so that cells having the same expression of biomarkers of the group are classified in the same class, wherein the group of biomarkers comprises includes a CD4 or CD8 T cell marker, and/or a B cell or plasma cell marker and/or a NK cell marker and/or a monocyte or dendritic cell marker, and at least 5 markers, typically at least 6, 7, 8, 9, 10, 11, 12, or at least 13 markers, selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
   (ii) determining the distribution of cells of a sample from the subject within the series of classes of the positive response control and/or the negative response control, the sample in the form of a sample of cells from blood of the subject;
   (iii) comparing the distribution of the cells of the sample from the subject within the series of classes with the negative response control and/or the positive response control, wherein the subject is determined to have a likelihood of being non-responsive to the treatment if the distribution of cells of the sample within the series of classes from the subject is substantially the same as the distribution of cells within the series of classes represented by the negative response control and/or is not the same as the distribution of cells within the series of classes represented by the positive response control.

In one embodiment, the group of biomarkers comprises at least 13 markers selected from CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1.

In one embodiment, the step (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
thereby determining the distribution of cells of the sample from the subject within the series of classes of the response controls.

In one embodiment, the step (iii) comparing the distribution of the cells of the sample from the subject within the series of classes comprises the step of:
comparing the measured number of cells of the sample from the subject in each class of the series of classes of the response controls with the number of cells of the response controls in each class of the series of classes of the response controls;
thereby determining whether the distribution of the cells of the sample within the series of classes of the response controls is the same as the distribution of cells represented by one of the response control.

As mentioned above, the inventors have found that the cells in a subject's blood which are associated with non-responsiveness to immune checkpoint therapy are ARG1′. The inventors have further characterised these cells and have found that they are a unique population of cells which exhibit a phenotype that is distinct from neutrophils, classical monocytes, non-classical monocytes, plasmacytoid dendritic cells and basophils. In this regard, the ARG1+ cells associated with non-responsiveness to immune checkpoint therapy comprise the phenotype ARG1+, CD14−, CD16−, CD66b−.

One aspect provides a method of determining whether a subject will be non-responsive to treatment with an immune checkpoint inhibitor comprising determining the level of cells which are ARG1+, CD14−, CD16−, CD66b−, in a blood sample from the subject;
wherein the subject is determined to be non-responsive to the treatment if the level of cells in the sample which are ARG1+, CD14−, CD16−, CD66b− is elevated relative to a positive response control.

A further aspect provides a method of treating cancer in a human subject, comprising:
(b) assessing whether the subject will be non-responsive to treatment with an immune checkpoint inhibitor comprising determining the level of cells which are ARG1+, CD14−, CD16−, CD66b−, in a blood sample from the subject;
(c) wherein the subject is determined to be non-responsive to the treatment if the level of cells in the sample which are ARG1+, CD14−, CD16−, CD66b− is elevated relative to a positive response control; and
   (b) if the subject is determined to be non-responsive to the treatment, administering an effective amount of an agent which inhibits arginase 1 activity and/or targets cells expressing arginase 1, for sufficient time to render the subject responsive to the immune checkpoint inhibitor; and
   (c) administering an effective amount of the immune checkpoint inhibitor, optionally in combination with further administration of the agent which inhibits arginase 1 activity and/or targets cells expressing arginase 1.

A further aspect provides a method of increasing responsiveness to treatment with an immune checkpoint inhibitor in a subject suffering from cancer that is non-responsive to the treatment, comprising administering to the subject an effective amount of an agent which inhibits arginase 1 activity, or targets cells expressing arginase 1, for sufficient time to render the subject responsive to the immune checkpoint inhibitor, wherein the subject has been determined to be non-responsive to the immune checkpoint inhibitor by
determining the level of cells which are ARG1+, CD14−, CD16−, CD66b− in a blood sample from the subject,
   wherein the subject is determined to be non-responsive to the treatment if the level of cells in the sample which are ARG1+, CD14−, CD16−, CD66b−, is elevated relative to a positive response control.

In one embodiment, the positive response control is the level of cells which are ARG1+, CD14−, CD16−, CD66b− in a blood sample from a subject or plurality of subjects that are responsive to treatment with the immune checkpoint inhibitor.

In various embodiments, the cells which are ARG1+, CD14−, CD16−, CD66b have the following phenotype:
(a) ARG1+, CD14−, CD16−, CD66b−, HLA-DR−;
(b) ARG1+, CD14−, CD16−, CD66b−, HLA-DR−, CD123+;
(c) ARG1+, CD14−, CD16−, CD66b−, HLA-DR−, CD123+, CD45RO+, CD38+;
(d) ARG1+, CD3−, CD14−, CD16−, CD66b−, CD19−, HLA-DR−, CD123+, CD45RO+, CD38+; (e) ARG1+, CD3−, CD14−, CD16−, CD66b−, CD19−, HLA-DR−, CD123+, CD45RO+, CD38+, CD304−;
(f) ARG1+, CD3−, CD14−, CD16−, CD66b−, CD19−, HLA-DR−, CD123+, CD45RO+, CD38+, CD304−, GM-CSFR alpha chain −;
(g) ARG1+, CD3−, CD14−, CD16−, CD66b−, CD19−, HLA-DR−, CD123+, CD45RO+, CD38+, CD304−, GM-CSFR alpha chain −, CD15−;
(h) ARG1+, CD3−, CD14−, CD16−, CD66b−, CD19−, HLA-DR−, CD123+, CD45RO+, CD38+, CD304−, GM-CSFR alpha chain −, CD15−, CD33lo;
(i) ARG1+, CD3−, CD14−, CD16−, CD66b−, CD19−, HLA-DR−, CD123+, CD45RO+, CD38+, CD304−, GM-CSFR alpha chain −, CD15−, CD33lo, CD192(CCR2)int;
(j) ARG1+, CD3−, CD14−, CD16−, CD66b−, CD19−, HLA-DR−, CD123+, CD45RO+, CD38+, CD304−, GM-CSFR alpha chain −, CD15−, CD33lo, CD45lo;
(k) ARG1+, CD3−, CD14−, CD16−, CD66b−, CD19−, HLA-DR−, CD123+, CD45RO+, CD38+, CD304−, GM-CSFR alpha chain −, CD15−, CD33lo, CD45lo, CD56−;
(l) ARG1+, CD3−, CD14−, CD16−, CD66b−, CD19−, HLA-DR−, CD123+, CD45RO+, CD38+, CD304−, GM-CSFR alpha chain −, CD15−, CD33lo, CD45lo, CD56−, CD20−.

A further aspect provides a method of treating cancer in a subject that is non-responsive to treatment with an immune checkpoint inhibitor, comprising administering to the subject an effective amount of an agent which inhibits arginase 1 activity, or targets cells expressing arginase 1, for sufficient time to render the subject responsive to the immune checkpoint inhibitor, for sufficient time to render the subject responsive to the immune checkpoint inhibitor, and thereafter administering an effective amount of the immune checkpoint inhibitor, optionally in combination with further administration of the agent which inhibits arginase 1 activity and/or targets cells expressing arginase 1.

As used herein, the terms “effective amount” refers to a dose of a drug or other agent that produces therapeutic effects for which it is administered. The exact dose will depend on the purpose of the treatment and will be ascertainable by one skilled in the art using known techniques (see, e.g., Remington: The Science and Practice of Pharmacy, 20th Edition, 2003, Gennaro, Ed., Lippincott, Williams & Wilkins). For example, an “effective amount” of an agent which inhibits arginase 1 activity and/or targets cells expressing arginase 1, will be an amount of the agent which is effective to render the subject responsive to the immune checkpoint inhibitor.

The agent which inhibits arginase 1 activity and/or targets cells expressing arginase 1, is administered for sufficient time to render the subject responsive to the immune checkpoint inhibitor.

The subject may be assessed as being responsive to the immune checkpoint inhibitor using, for example, the methods described in WP2019/051542.

In one embodiment, the subject may be assessed as being responsive to the immune checkpoint inhibitor following treatment with the agent which inhibits arginase 1 activity and/or targets cells expressing arginase 1 by a method comprising:
determining the distribution of cells of a sample from the subject who has been administered the agent within the series of classes of a positive response control and/or a negative response control, the sample in the form of a sample of cells from blood of the subject; and
comparing the distribution of the cells of the sample from the subject within the series of classes with the negative response control and/or the positive response control, wherein the subject is determined to have a likelihood of being responsive to the treatment if the distribution of cells of the sample within the series of classes from the subject is substantially the same as the distribution of cells within the series of classes represented by the positive response control and/or is not the same as the distribution of cells within the series of classes represented by the negative response control.

The length of time necessary for administration of the agent which inhibits arginase 1 activity and/or targets cells expressing arginase 1, may therefore be monitored by assessing at intervals after administration of the agent.

Agents which inhibits arginase 1 activity and/or targets cells expressing arginase 1, are known in the art. Examples of such agents which inhibit arginase activity include, for example, N-hydroxy-L-arginine (NOHA), 2(S)-amino-6-boronohexanoic acid (ABH), N-hydroxy-nor-L-arginine (nor-NOHA), α-difluoromethylornithine (DFMO), L-norvaline, iodoacetyl-L-ornithine, iodoacetyl-L-lysine, L-lysine, and L-citrulline, (2s)-(+)-amino-5-iodoacetamidopentanoic acid, NG-hydroxy-L-arginine, (2S)-(+)-amino-6-iodoacetamidohexanoic acid, and (R)-2-amino-6-borono-2-(2-(piperidin-1-yl)ethyl)hexanoic acid, CB-1158 (numidargistat), OATD-02 (patent no. WO2017191130A4), or compounds described in WO2019173188A1, WO2019177873A1, and WO2020161675A1.

In one embodiment, the agent inhibits intracellular arginase 1 and/or targets cells expressing high levels of intracellular arginase 1.

In one embodiment, the agent which targets cells expressing arginase 1 targets cells constitutively expressing arginase 1.

In embodiments in which the agents target cells expressing arginase, the agent may be an antibody which specifically binds, and typically inactivates, cells expressing arginase 1.

In another embodiment, the agent which inhibits arginase 1 activity and/or targets cells expressing arginase 1 is an agent which reduces or inhibits arginase gene expression, through either inhibition of transcription or translation. For example, the agent which inhibits arginase activity may be, for example, a gene silencing nucleic acid which targets arginase mRNA, such as for example siRNA agents (see for example, WOO/44895, WO99/32619, WO01/75164, WO01/92513, WO01/29058, WO01/89304, WO02/16620, and WO02/29858), CRISPR agents (see, for example, Jinek et. al., Science. 2012 Aug. 17; 337(6096):816-21; Qi et al., Cell. 2013 Feb. 28; 152(5):1173-83), or antisense RNA.

Typically, dosages of the agent which inhibits arginase activity in the ARG1+ cells or which reduces or inhibits arginase gene expression which may be administered to a subject, preferably a human, range in amount from 1 μg to about 100 g per kilogram of body weight of the subject. While the precise dosage administered will vary depending upon any number of factors, including for example, the type of cancer state being treated, the nature of the agent (e.g., small molecule, siRNA, or antibody), the metabolic stability and length of action of the agent, the age, body weight, general health, sex, diet, mode and time of administration, rate of excretion, drug combination, the severity of the particular condition, and the subject undergoing therapy.

The agent may be administered as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

As used herein, an ‘immune check-point inhibitor’ is a molecule or agent which inhibits the immune suppression by an immune checkpoint. The check-point inhibitor may be an inhibitor of any checkpoint which suppresses T cell function and is used for cancer treatment. In one embodiment, the checkpoint inhibitor is an inhibitor of the interaction between PD-1 and its ligand. In one embodiment, the checkpoint inhibitor that is an inhibitor of the interaction between PD-1 and its ligand is any anti-PD-1 or anti-PD-L1 antibody. Examples of anti-PD-1 antibodies used in cancer therapy include pembrolizumab, nivolumab or cemiplimab. Examples of anti-PD-L1 antibodies include atezolizumab, avelumab, and durvalumab.

In another embodiment, the checkpoint inhibitor is an inhibitor of the binding of CTLA-4 and its ligand. In one embodiment, the checkpoint inhibitor that is an inhibitor of the interaction between CTLA-4 and its ligand is an anti-CTLA-4 antibody. An example of anti-CTLA-4 antibody used in cancer therapy is ipilimumab.

Other examples of check-point inhibitors include inhibitors of A2AR, CD276 (B7-H3), VTCN1 (B7-H4), IDO (Indoleamine 2,3-dioxygenase), KIR (killer-cell immunoglobulin-like receptor), LAG 3 (lymphocyte activation gene-3), TIM-3 (T-cell immunoglobulin domain and mucin domain 3) and VISTA (V-domain Ig suppressor of T cell activation).

The cancer may be any cancer which is treatable with an immune checkpoint inhibitor in a subject that is responsive to such treatment. Examples of cancers that may be treated using the methods described herein include bladder cancer, colorectal cancer, breast cancer, prostate cancer, renal cancer, hepatocellular cancer, lung cancer, ovarian cancer, urothelial cancer, cervical cancer, gastric cancer, esophageal cancer, head and neck cancer, melanoma, squamous cell carcinoma, Merkel cell carcinoma, mesothelioma, non-small cell lung cancer (NSCLC), small-cell lung cancer, colon cancer; multiple myeloma (MM), Hodgkin's lymphoma (HL), B-cell lymphoma or diffuse large B-cell lymphoma (DLBCL).

Another aspect provides a pharmaceutical composition comprising an agent which inhibits arginase 1 activity and/or targets cells expressing arginase 1, an immune checkpoint inhibitor, and a pharmaceutically acceptable carrier.

A “pharmaceutically acceptable carrier” means that it is compatible with the other ingredients of the composition and is not deleterious to a subject. The compositions may contain other therapeutic agents as described below, and may be formulated, for example, by employing conventional liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

The pharmaceutical compositions are typically in the form of a sterile injectable aqueous suspension. This suspension may be formulated according to the known art and contain the active materials in admixture with excipients suitable for the manufacture of aqueous suspensions. Such excipients may include suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectable formulations.

A further aspect provides a kit for the treatment of cancer in a subject, comprising:
(a) an agent which inhibits arginase activity in ARG1+ cells, or targets cells expressing arginase; and optionally
(b) an immune checkpoint inhibitor.
   The kit may further comprise instructions for use.

Generally, the term “treating” means affecting a subject, tissue or cell to obtain a desired pharmacological and/or physiological effect and include: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease. In one embodiment, treatment achieves the result of reducing the number of cancer cells in the recipient subject.

The term “subject” refers to any animal having a disease which requires treatment by the present method. In the context of the present disclosure, the subject is a human.

It is to be understood that the terminology used herein is for the purpose of describing particular embodiments only and is not intended to limit the scope of the present invention which will be limited only by the appended claims. As used herein and in the appended claims, the singular forms “a”, “an”, and “the” include plural reference unless the context clearly indicates otherwise. Thus, for example, a reference to “an antibody” includes a plurality of such antibodies. Unless defined otherwise, all technical and scientific terms used herein have the same meaning as commonly understood by one of ordinary skill in the art to which this invention belongs.

Although any materials and methods similar or equivalent to those described herein can be used to practice or test the present invention, preferred materials and methods are described herein.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

In order to exemplify the nature of the present invention such that it may be more clearly understood, the following non-limiting examples are provided.

### EXAMPLES

A peripheral blood sample is obtained using venepuncture, with the blood collected into a tube that prevents clotting (for example, one containing EDTA or heparin). The leucocytes are purified using Ficoll-Hypaque density gradient centrifugation to isolate peripheral blood mononuclear cells (PBMCs) then prepared immediately for cytometry, or cryopreserved for long-term storage, for example at −80° C. or in liquid nitrogen. Cryopreserved leucocytes are thawed before analysis using conventional techniques.

The distribution of cells is determined using a technique that identifies expression of pre-determined proteins or transcripts within each single cell within the sample. Typically, the cells are treated with a mixture of between 10 and 40 antibodies, each specific for an individual cell protein and labelled with a unique metal isotope or fluorochrome such that the treated sample can be analysed using mass cytometry or fluorescence cytometry, respectively.

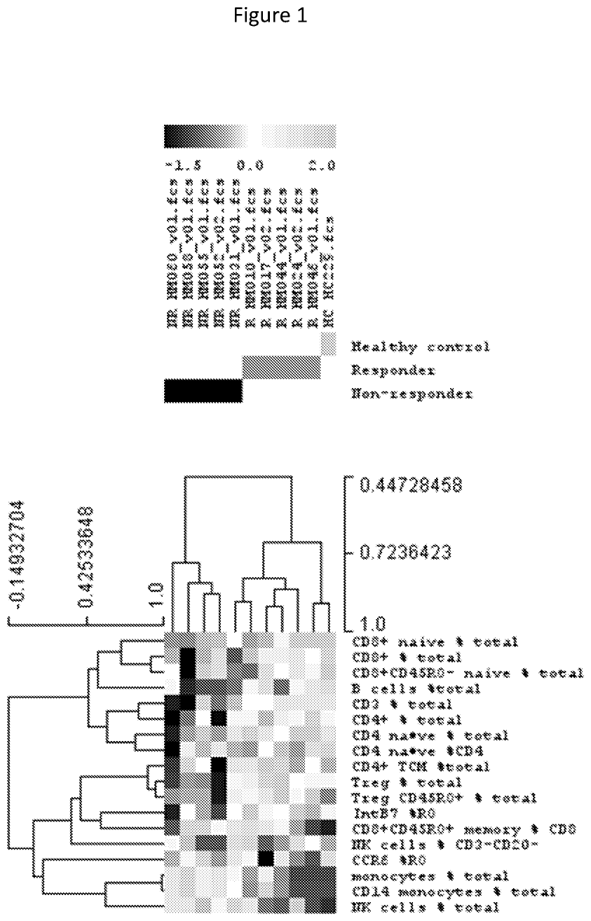

In a cohort of 10 stage 4 non-small cell lung cancer (NSCLC) patients scheduled for anti-PD-1/PD-L1 therapy, pre-therapy PBMCs were analysed by means of mass cytometry. To determine the immune signature, the data from 13 mAbs out of the panel of 41 (antibodies specific for CD45, CD3, CD4, CD8, CD45RO, CD197 (CCR7), CD25, CD127, CD196 (CCR6), Integrin beta7, CD56, CD16, and CD14) was analysed. 5 patients made clinical responses to anti-PD-1/PD-L1 therapy and in 5 patients disease progressed despite anti-PD-1/PD-L1 therapy. The pre-therapy blood immune signature to anti-PD-1/PD-L1 therapy is shown in FIG. 1. Hierarchical clustering confirmed the utility of the pre-therapy blood signature in distinguishing responder and non-responder patients.

Table 2 is a definition of the cell populations depicted in FIG. 1. Cytometric analysis with a panel of 13 mAbs (specific for (specific for CD45, CD3, CD4, CD8, CD45RO, CD197 (CCR7), CD25, CD127, CD196 (CCR6), Integrin beta7, CD56, CD16, and CD14) defined 14 cell populations that define the immune signature of clinical non-response to immunotherapy directed against the PD-1/PD-L1 pathway.

TABLE 2

| Designation of | Antibodies to 13 specificities required to define the cell populations in the immune signature | | | | | | | | | | | | |
|---|---|---|---|---|---|---|---|---|---|---|---|---|---|
| cell population | | | | | | | | | | Integrin | | | |
| in FIG. 1 | CD45 | CD3 | CD4 | CD8 | CD45RO | CCR7 | CD25 | CD127 | CCR6 | B7 | CD14 | CD16 | CD56 |
| CD8+ naïve % total | positive | positive | negative | positive | negative | NA | NA | NA | NA | NA | negative | NA | NA |
| CD8+ % total | positive | positive | negative | positive | NA | NA | NA | NA | NA | NA | negative | NA | NA |
| CD8+CD45RO− | positive | positive | negative | positive | negative | positive | negative | NA | NA | NA | negative | NA | NA |
| naïve % total | | | | | | | | | | | | | |
| B cells % total | positive | negative | negative | negative | NA | NA | NA | NA | positive | NA | negative | negative | negative |
| CD3+ % total | positive | positive | NA | NA | NA | NA | NA | NA | NA | NA | NA | NA | NA |
| CD4+ % total | positive | positive | positive | negative | NA | NA | NA | NA | NA | NA | negative | NA | NA |
| CD4+ naïve % total | positive | positive | positive | negative | negative | positive | NA | NA | NA | NA | negative | NA | NA |
| CD4+ naïve % CD4 | positive | positive | positive | negative | negative | positive | NA | NA | NA | NA | negative | NA | NA |
| CD4+ TCM % total | positive | positive | positive | negative | positive | positive | NA | NA | NA | NA | negative | NA | NA |
| Treg % total | positive | positive | positive | negative | NA | NA | positive | negative | NA | NA | negative | NA | NA |
| Treg CD45RO+ | positive | positive | positive | negative | positive | NA | positive | negative | NA | NA | negative | NA | NA |
| % total | | | | | | | | | | | | | |
| Beta7+ of | positive | positive | positive | negative | positive | NA | NA | NA | NA | positive | negative | NA | NA |
| CD45RO+CD4+ | | | | | | | | | | | | | |
| CD8+CD45RO+ | positive | positive | negative | positive | positive | NA | NA | NA | NA | NA | negative | NA | NA |
| memory % CD8+ | | | | | | | | | | | | | |
| NK cells % | positive | negative | negative | negative | NA | NA | NA | NA | NA | NA | negative | positive | positive |
| CD3−CD20− | | | | | | | | | | | | | |
| CCR6+ of | positive | positive | positive | negative | positive | NA | NA | NA | positive | NA | negative | NA | NA |
| CD45RO+CD4+ | | | | | | | | | | | | | |
| Monocytes % total | positive | negative | low | negative | NA | NA | NA | NA | NA | NA | positive | positive | negative |
| CD14+CD16− | positive | negative | negative | negative | NA | NA | NA | NA | NA | NA | positive | negative | negative |
| classical monocytes | | | | | | | | | | | | | |
| % total | | | | | | | | | | | | | |
| NK cells % total | positive | negative | negative | negative | NA | NA | NA | NA | NA | NA | negative | positive | positive |

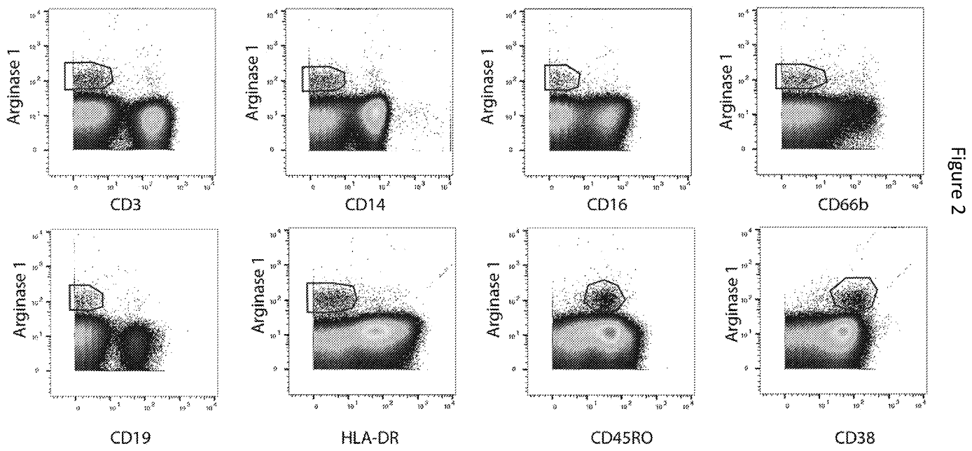

Intracellular detection of arginase 1 using a metal conjugated mAb specific for human arginase 1 revealed a distinct population of arginase 1 expressing cells that were clearly identified within the total PBMC population, as shown in FIG. 2. Cells constitutively expressing high level arginase 1 in pre-therapy PBMCs from NSCLC patients were negative for expression of HLA-DR, CD14, CD16 and CD66b, and positive for CD45RO and CD38. Although monocytic-MDSCs (M-MDSCs, HLA-DRlo CD14+) and neutrophilic (PMN-MDSCs, HLA-DRlo CD16+CD66b+) are reported to express arginase 1, no expression of intracellular arginase 1 was detected within M-MDSCs or PMN-MDSCs within the unstimulated pre-therapy blood samples examined in this study. Thus the constitutive expression of intracellular arginase 1 within circulating blood cells cannot be accounted for by M-MDSCs or PMN-MDSCs described in previous studies.

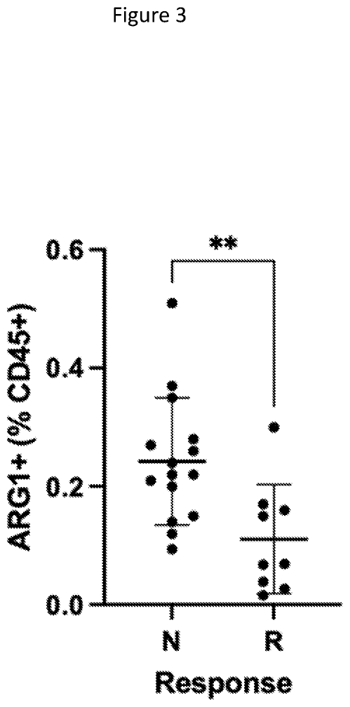

A statistically significant negative correlation was found between the number of cells expressing intracellular arginase 1 in pre-therapy PBMCs and clinical response to anti-PD-1/PD-L1 therapy in 24 NSCLC patients (FIG. 3).

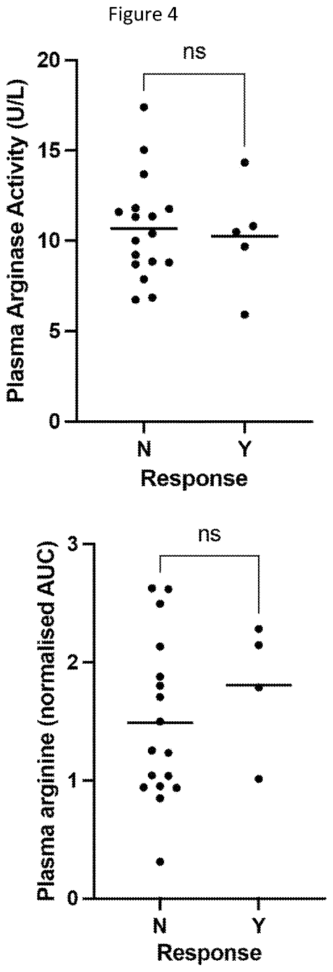

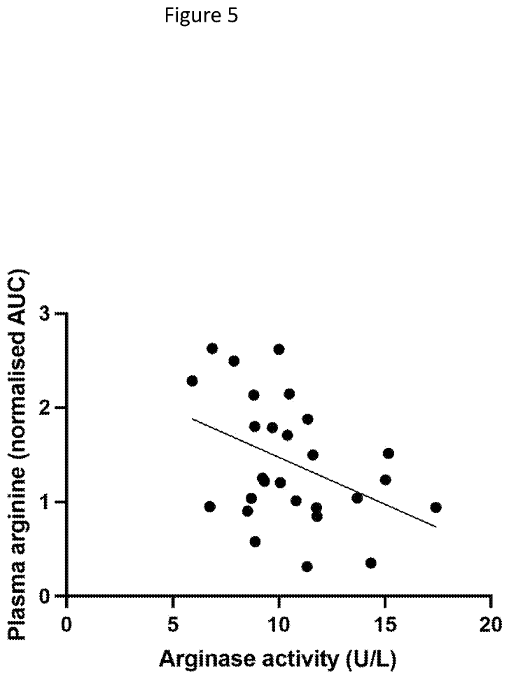

In a cohort of 21 NSCLC patients for whom pre-therapy plasma samples were available, we measured plasma arginase activity (using a colorimetric assay that measured conversion of L-arginine to L-ornithine) and plasma L-arginine (using mass spectrometry). There was no statistically significant difference between responder and non-responder patents (FIG. 4), although as expected plasma arginine activity was higher in samples in which the arginine content was lower (FIG. 5). These results strongly support a model in which arginase expressed within cells is the major driver of the compromised the T cell response to anti-PD-1/PD-L1 therapy to the extent that an effective clinical response cannot be mounted.

These data indicate that therapies that target the cells making arginase, rather than arginase within the circulation, will be of benefit in converting non-responder patients to responder status.

Markers Associated with Arginase 1 Expressing Cells

A panel of monoclonal antibodies were used to obtain a full definition of arginase 1 expressing cell phenotype and how these cells are distinct from other circulating myeloid cells. The results are shown in Table 3.

TABLE 3
Differences in marker expression between arginase 1-expressing cells,
neutrophils, classical monocytes and non-classical monocytes.

| | arginase 1 | | classical | non-classical |
|---|---|---|---|---|
| | expressing | Neutrophils | monocytes and | monocytes and |
| Biomarker | cells | and pmn-MDSCs | mo-MDSCs | mo-MDSCs |
| arginase 1 | positive | negative | negative | negative |
| CD123 (IL-3Ralpha chain) | positive | negative | negative | negative |
| GM-CSFR alpha chain | negative | positive | positive | positive |
| CD192 (CCR2) | int | low | high | int to low |
| CD11b | int | high | high | high |
| CD33 | low | positive | high | high |
| HLA-DR | negative | low | positive | positive |
| CD304 | negative | negative | negative | negative |
| CD45 | lo | lo | int-high | high |
| CD38 | very high | low | high | high |
| CD14 | negative | negative | positive | variable |
| CD16 | negative | positive | negative | positive |
| CD15 | negative | positive | negative | negative |
| CD66b | negative | positive | negative | negative |
| CD3 | negative | negative | negative | negative |
| CD4 | negative | negative | low | low |
| CD8a | negative | negative | negative | negative |
| CD19/CD20 | negative | negative | negative | negative |
| CD56 | negative | negative | negative | negative |

Arginase 1 expression in myeloid-derived suppressor cells (MDSCs) has been described in the literature, and is documented in both neutrophilic PMN-MDSCs and monocytic mo-MDSCs. The arginase 1-expressing cells described herein are distinct from both types of MDSCs: they are negative for CD14, used to identify mo-MDSCs and negative for CD15, CD16 and CD66b, used to identify PMN-MDSCs. They express CD123, which is not expressed by MDSCs. CD123 is also highly expressed by plasmacytoid DCs (pDCs). The arginase 1-expressing cells described herein are distinct from pDCs because they are negative for HLA-DR, which is expressed by pDCs. CD123 is also highly expressed by basophils. The arginase 1-expressing cells described herein are distinct from basophils because basophils do not express arginase 1.

In a further experiment to assess the morphology of arginase 1-expressing cells, arginase 1-expressing PBMC from a NSCLC patient were sorted using a combination of mAbs to CD45, HLA-DR, CD11b, CD123, arginase 1, CD14, CD15, lineage (CD19, CD3, CD56). For comparison, pDCs, neutrophils and monocytes were also sorted. Sorted cells were cytospun onto glass slides and stained with Giemsa. Analysis of nuclear morphology indicated that the arginase 1-positive cells had an unusual morphology, with a multilobed nucleus, usually with 2 or 3 lobes. The nuclei of many arginase 1-expressing cells showed a cerebriform appearance, while a small number were cleaved in appearance. The cells did not contain the basophilic granules typical of basophils. A second population of CD123+ HLA-DR− arginase 1-negative cells (0.23% of CD45+ cells) were smaller in size and had a lymphoid appearance. They likely correspond to the ILC-like population described by Mora-Velandia et al, 2017, Front. Immunol. 8:176 in the context of psoriasis.

In addition to NSCLC in which arginase 1-expressing cells in the blood are more frequent than in healthy controls and correlate with primary resistance to immunotherapy treatment targeting the PD-1/PD-L1 pathway, the presence of abnormally high numbers of these cells was also demonstrated in patients with colorectal cancer. Arginase 1-expressing cells were not present within the mixture of cells from the dissociated cancer samples from the same patients.

### Targeting Arginase 1-Positive Cells to Restore and Reverse Non-Responsiveness to Anti-PD-1/PD-L1 Therapy

We propose that the use of agents that target arginase 1-positive cells will, over time, revert the immune signature of non-response back to normal. However, it may not be necessary to allow enough time to revert the signature before anti-PD-1/PD-L1 therapy is commenced, if arginase 1-targeted therapy allows potential cancer-controlling T cells to regain normal signalling function, even before other aspects of the immune signature, such as the deficit in naïve T cells, have normalised.

Also described herein are the following items 1 to 20:
1. A method of treating cancer in a human subject, comprising:
(a) assessing whether the subject will be non-responsive to treatment with an immune checkpoint inhibitor comprising:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 13 markers selected from CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase 1;
   (ii) determining the distribution of cells in a blood sample from the subject within the series of classes of the positive response control and the negative response control;
   (iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control; and
(b) if the subject is determined to be non-responsive to the treatment, administering an effective amount of an agent which inhibits arginase 1 activity and/or targets cells expressing arginase 1, for sufficient time to render the subject responsive to the immune checkpoint inhibitor; and
(c) administering an effective amount of the immune checkpoint inhibitor, optionally in combination with further administration of the agent which inhibits arginase 1 activity and/or targets cells expressing arginase 1.
2. The method of item 1, wherein the group of biomarkers comprises the markers CD45, CD45RO, CD3, CD4, CD8, CD14, CD16, CD127, CD25, CD56, CD196 (CCR6), CD197 (CCR7), and Integrin beta7.
3. The method of item 1 or 2, wherein (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
   thereby determining the distribution of cells of the sample from the subject within the series of classes of the response controls.
4. The method of item 1, wherein (iii) comparing the distribution of the cells of the sample from the subject within the series of classes comprises the step of:
   comparing the measured number of cells of the sample from the subject in each class of the series of classes of the response controls with the number of cells of the response controls in each class of the series of classes of the response controls;
   thereby determining whether the distribution of the cells of the sample within the series of classes of the response controls is the same as the distribution of cells represented by one of the response control.
5. The method of any one of items 1 to 4, wherein the sample is a peripheral blood sample.
6. The method of any one of items 1 to 5, further comprising monitoring the effectiveness of the treatment comprising the step of determining comparing the level of cells expressing intracellular arginase 1 in a blood sample from the subject after the treatment to the level of cells expressing intracellular arginase 1 before the treatment.
7. A method of increasing responsiveness to treatment with an immune checkpoint inhibitor in a subject suffering from cancer that is non-responsive to the treatment, comprising administering to the subject an effective amount of an agent which inhibits arginase 1 activity, or targets cells expressing arginase 1, for sufficient time to render the subject responsive to the immune checkpoint inhibitor, wherein the subject has been determined to be non-responsive to the immune checkpoint inhibitor by:
(a) assessing the likelihood that the subject will be non-responsive to treatment with an immune checkpoint inhibitor comprising:
   (i) providing a positive response control from one or more positive responders who have formed a clinical response to the treatment, and a negative response control from one or more non-responders who have not formed a clinical response to the treatment, each response control representing a distribution of cells of a blood sample from the positive responder and the non-responder within a series of classes, wherein the distribution of cells is according to the expression by each cell of the sample of a group of biomarkers so that cells having the same expression of biomarkers of the group are classified in the same class, wherein the group of biomarkers comprises includes a CD4 or CD8 T cell marker, and/or a B cell or plasma cell marker and/or a NK cell marker and/or a monocyte or dendritic cell marker, and at least 13 markers selected from CD45, CD45RA, CD45RO, CD3, CD4, CD8, CD19, CD20, CD14, CD16, CD11b, CD11c, CD66b, CD304, FoxP3, CD127, CD25, CD56, HLA-DR, IgD, CD27, CD86, FCRL3, CD274 (PD-1), CD279 (PD-L1), TIGIT, CD38, Ki67, Granzyme B, CD134 (OX40), CD194 (CCR4), CD195 (CCR5), CD196 (CCR6), CD197 (CCR7), Integrin beta7, CLA, CD183 (CXCR3), CD185, (CXCR5), CCR10 and intracellular arginase;
   (ii) determining the distribution of cells of a sample from the subject within the series of classes of the positive response control and the negative response control, the sample in the form of a sample of cells from blood of the subject;
   (iii) comparing the distribution of the cells of the sample from the subject within the series of classes with the negative response control and/or the positive response control, wherein the subject is determined to have a likelihood of being non-responsive to the treatment if the distribution of cells of the sample within the series of classes from the subject is substantially the same as the distribution of cells within the series of classes represented by the negative response control and/or is not the same as the distribution of cells within the series of classes represented by the positive response control.
8. The method of item 7, wherein the group of biomarkers comprises a CD4 or CD8 T cell marker, and/or a B cell or plasma cell marker and/or a NK cell marker and/or a monocyte or dendritic cell marker, and at least 13 markers selected from CD45, CD45RO, CD3, CD4, CD8, CD14, CD16, CD127, CD25, CD56, CD196 (CCR6), CD197 (CCR7), and Integrin beta7.
9. The method of item 7 or 8, wherein (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
   thereby determining the distribution of cells of the sample from the subject within the series of classes of the response controls.
10. The method of item 7, wherein (iii) comparing the distribution of the cells of the sample from the subject within the series of classes comprises the step of:
   comparing the measured number of cells of the sample from the subject in each class of the series of classes of the response controls with the number of cells of the response controls in each class of the series of classes of the response controls;
   thereby determining whether the distribution of the cells of the sample within the series of classes of the response controls is the same as the distribution of cells represented by one of the response control.
11. The method of any one of items 7 to 10, wherein the sample is a peripheral blood sample.
12. The method of any one of items 7 to 11, further comprising monitoring the effectiveness of the treatment comprising the step of determining comparing the level of cells expressing intracellular arginase 1 in a blood sample from the subject after the treatment to the level of cells expressing intracellular arginase before the treatment.
13. A method of treating cancer in a subject that is non-responsive to treatment with an immune checkpoint inhibitor, comprising administering to the subject an effective amount of an agent which inhibits arginase activity, or targets cells expressing arginase, for sufficient time to render the subject responsive to the immune checkpoint inhibitor, and administering an effective amount of the immune checkpoint inhibitor, optionally in combination with further administration of the agent which inhibits arginase 1 activity and/or targets cells expressing arginase 1.
14. The method of any one of items 1 to 13, wherein the immune checkpoint inhibitor is an inhibitor of the interaction between PD-1 and its ligand.
15. The method of any one of items 1 to 14, wherein, the immune checkpoint inhibitor is an anti-PD-1 antibody or anti-PD-L1 antibody.
16. The method of any one of items 1 to 15, wherein the immune checkpoint inhibitor is an anti-PD-1 monoclonal antibody, or an anti-PD-L1 monoclonal antibody.
17. The method of any one of items 1 to 16, wherein the immune checkpoint inhibitor is nivolumab, pembrolizumab, cemiplimab, durvalumab, atezolizumab or avelumab.
18. The method of any one of items 1 to 17, wherein the agent which targets cells expressing arginase 1 targets cells constitutively expressing arginase 1.
19. A pharmaceutical composition comprising an agent which inhibits arginase 1 activity, or targets cells expressing arginase 1, and an immune checkpoint inhibitor.
20. A kit for the treatment of cancer, comprising:
(a) an agent which inhibits arginase 1 activity, or targets cells expressing arginase 1; and optionally
(b) an immune checkpoint inhibitor.

The present application claims priority from Australian provisional patent application no. 2022903578, the entirety of which is incorporated herein by reference.

1.-68. (canceled)

69. A method of treating cancer in a subject that has been identified as being non-responsive to treatment with an immune checkpoint inhibitor, comprising:
(a) administering an effective amount of an agent which inhibits arginase 1 activity and/or targets cells expressing arginase 1, for sufficient time to render the subject responsive to the immune checkpoint inhibitor; and
(b) administering an effective amount of the immune checkpoint inhibitor, optionally in combination with further administration of the agent which inhibits arginase 1 activity and/or targets cells expressing arginase 1 wherein the subject has been identified as non-responsive to the immune checkpoint inhibitor by
(i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises at least 5 markers selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, GM-CSFR alpha chain, and intracellular arginase 1;
(ii) determining the distribution of cells in a blood sample from the subject within the series of classes of the positive response control and the negative response control;
(iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

70. The method of claim 69, the group of biomarkers comprises at least 13 markers selected from CD45, CD45RO, CD3, CD4, CD8, CD19, CD20, CD14, CD15, CD16, CD11b, CD66b, CD304, CD123, CD127, CD25, CD33, CD56, HLA-DR, CD38, CD192 (CCR2), CD196 (CCR6), CD197 (CCR7), Integrin beta7, GM-CSFR alpha chain, and intracellular arginase 1.

71. The method of claim 69, wherein the group of biomarkers comprises HLA-DR, CD14, CD16, CD66b, CD45RO, CD38 and intracellular arginase 1.

72. The method of claim 69, wherein the group of biomarkers comprises HLA-DR, CD3, CD14, CD16, CD19, CD66b, CD45RO, CD38 and intracellular arginase 1.

73. The method of claim 69, wherein the group of biomarkers comprises HLA-DR, CD14, CD16, CD66b, and intracellular arginase 1.

74. The method of claim 69, wherein (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
thereby determining the distribution of cells of the sample from the subject within the series of classes of the response controls.

75. The method of claim 69, wherein (iii) comparing the distribution of the cells of the sample from the subject within the series of classes comprises the step of:
comparing the measured number of cells of the sample from the subject in each class of the series of classes of the response controls with the number of cells of the response controls in each class of the series of classes of the response controls;
thereby determining whether the distribution of the cells of the sample within the series of classes of the response controls is the same as the distribution of cells represented by one of the response control.

76. The method of claim 69, wherein the sample is a peripheral blood sample.

77. The method of claim 69, wherein the immune checkpoint inhibitor is an inhibitor of the interaction between PD-1 and its ligand.

78. The method of claim 69, further comprising monitoring the effectiveness of the treatment comprising the step of comparing the level of cells expressing intracellular arginase 1 in a blood sample from the subject after the treatment to the level of cells expressing intracellular arginase 1 before the treatment.

79. The method of claim 69, further comprising after step (a) of assessing whether the subject is responsive or non-responsive to treatment with the immune checkpoint inhibitor.

80. The method of claim 69, wherein the cancer is selected from bladder cancer, colorectal cancer, breast cancer, prostate cancer, renal cancer, hepatocellular cancer, lung cancer, ovarian cancer, urothelial cancer, cervical cancer, gastric cancer, esophageal cancer, head and neck cancer, melanoma, squamous cell carcinoma, Merkel cell carcinoma, mesothelioma, non-small cell lung cancer (NSCLC), small-cell lung cancer, colon cancer; multiple myeloma (MM), Hodgkin's lymphoma (IL), B-cell lymphoma or diffuse large B-cell lymphoma (DLBCL)

81. A method of determining whether a subject suffering from cancer will be non-responsive to treatment with an immune checkpoint inhibitor, the method comprising:
(i) providing a positive response control from one or more positive responders who have formed a clinical response to the treatment, and a negative response control from one or more non-responders who have not formed a clinical response to the treatment, each response control representing a distribution of cells of a blood sample from the positive responder(s) and the non-responder(s) within a series of classes, wherein the distribution of cells is according to the expression by cells of the sample of a group of biomarkers so that cells having the same expression of biomarkers from the group of biomarkers are classified in the same class, wherein the group of biomarkers comprises intracellular arginase 1 and at least 4 markers selected from CD45, CD45RA, CD45RO, CD3, CD4, CD8, CD19, CD20, CD14, CD15, CD16, CD11b, CD11c, CD66b, CD304, FoxP3, CD123, CD127, CD25, CD33, CD56, HLA-DR, IgD, CD27, CD86, FCRL3, CD274 (PD-1), CD279 (PD-L1), TIGIT, CD38, Ki67, Granzyme B, CD134 (OX40), CD192 (CCR2), CD194 (CCR4), CD195 (CCR5), CD196 (CCR6), CD197 (CCR7), Integrin beta7, CLA, CD183 (CXCR3), CD185, (CXCR5), CCR10, and GM-CSFR alpha chain;
(ii) determining the distribution of cells in a blood sample from the subject within the series of classes of the positive response control and the negative response control;
(iii) comparing the distribution of the cells of the sample from the subject within the series of classes with the negative response control and/or the positive response control, wherein the subject is determined to be non-responsive to the treatment if the distribution of cells of the sample from the subject within the series of classes is substantially the same as the distribution of cells within the series of classes represented by the negative response control and/or is not the same as the distribution of cells within the series of classes represented by the positive response control.

82. The method of claim 81, wherein the group of biomarkers comprises intracellular arginase 1 and CD3, HLA-DR, CD14, CD16, CD19, CD66b, CD45RO and CD38.

83. The method of claim 81, wherein the group of biomarkers comprises intracellular arginase 1 and CD3, CD45, HLA-DR, CD11b, CD123, CD14, CD15, CD19, and CD56.

84. The method of claim 81, wherein (ii) determining the distribution of cells of a sample within the series of classes comprises the steps of:
a. assessing each cell of the sample from the subject for the expression of each biomarker of the group of biomarkers of the response controls;
b. classifying each cell of the sample from the subject into one of the series of classes of the response controls;
c. measuring the number of cells of the test sample in each class of the series of classes of the response controls;
thereby determining the distribution of cells of the sample from the subject within the series of classes of the response controls.

85. The method of claim 81, wherein (iii) comparing the distribution of the cells of the sample from the subject within the series of classes comprises the step of:
comparing the measured number of cells of the sample from the subject in each class of the series of classes of the response controls with the number of cells of the response controls in each class of the series of classes of the response controls;
thereby determining whether the distribution of the cells of the sample within the series of classes of the response controls is the same as the distribution of cells represented by one of the response control.

86. The method of claim 81, wherein the sample is a peripheral blood sample.

87. The method of claim 81, wherein the cancer is selected from bladder cancer, colorectal cancer, breast cancer, prostate cancer, renal cancer, hepatocellular cancer, lung cancer, ovarian cancer, urothelial cancer, cervical cancer, gastric cancer, esophageal cancer, head and neck cancer, melanoma, squamous cell carcinoma, Merkel cell carcinoma, mesothelioma non-small cell lung cancer (NSCLC), small-cell lung cancer, colon cancer; multiple myeloma (MM), Hodgkin's lymphoma (HL), B-cell lymphoma or diffuse large B-cell lymphoma (DLBCL).

88. The method of claim 81, further comprising treating the subject, wherein the treating comprises:
(a) administering an effective amount of an agent which inhibits arginase 1 activity and/or targets cells expressing arginase 1, for sufficient time to render the subject responsive to the immune checkpoint inhibitor, if the subject is determined to be non-responsive to the treatment; and/or
(b) administering an effective amount of the immune checkpoint inhibitor, optionally in combination with administration of an agent which inhibits arginase 1 activity and/or targets cells expressing arginase 1.

89. The method of claim 88, wherein the immune checkpoint inhibitor is an inhibitor of the interaction between PD-1 and its ligand.

90. The method of claim 88, further comprising after step (a) of assessing whether the subject is responsive or non-responsive to treatment with the immune checkpoint inhibitor.

91. The method of claim 88, further comprising monitoring the effectiveness of the treatment comprising the step of comparing the level of cells expressing intracellular arginase 1 in a blood sample from the subject after the treatment to the level of cells expressing intracellular arginase 1 before the treatment.

92. A method of determining whether a subject will be non-responsive to treatment with an immune checkpoint inhibitor comprising determining the level of cells which are ARG1+, CD14−, CD16−, CD66b−, in a blood sample from the subject;
wherein the subject is determined to be non-responsive to the treatment if the level of cells in the sample which are ARGT+, CD14−, CD16−, CD66b− is elevated relative to a positive response control.

93. The method of claim 89, wherein the positive response control is the level of cells which are ARGT+, CD14−, CD16−, CD66b− in a blood sample from a subject or plurality of subjects that are responsive to treatment with the immune checkpoint inhibitor.

94. The method of claim 92, wherein the ARGT+, CD14−, CD16−, CD66b− cells are ARGT+, CD14−, CD16−, CD66b−, HLA-DR−.

95. The method of claim 92, wherein the ARGT+, CD14−, CD16−, CD66b− cells are ARGT+, CD14−, CD16−, CD66b−, HLA-DR−, CD123+.

96. The method of claim 92, wherein the ARG1+, CD14−, CD16−, CD66b− cells are ARG1+, CD14−, CD16−, CD66b−, HLA-DR−, CD123+, CD45RO+, CD38+.

97. The method of claim 92, wherein the ARG1+, CD14−, CD16−, CD66b− cells are ARGT+, CD14−, CD16−, CD66b−, HLA-DR−, CD45RO+, CD38+, CD19−, CD3−.

98. The method of claim 89, wherein the ARGT+, CD14−, CD16−, CD66b− cells are ARGT+, CD3−, CD14−, CD16−, CD66b−, CD19−, HLA-DR−, CD45RO+CD38+.

99. The method of claim 92, further comprising treating the subject, wherein the treating comprises:
(a) administering an effective amount of an agent which inhibits arginase 1 activity and/or targets cells expressing arginase 1, for sufficient time to render the subject responsive to the immune checkpoint inhibitor, if the subject is determined to be non-responsive to the treatment; and/or
(b) administering an effective amount of the immune checkpoint inhibitor, optionally in combination with administration of an agent which inhibits arginase 1 activity and/or targets cells expressing arginase 1.

100. The method of claim 99, further comprising after step (a) of assessing whether the subject is responsive or non-responsive to treatment with the immune checkpoint inhibitor.

101. The method of claim 92, wherein the immune checkpoint inhibitor is an inhibitor of the interaction between PD-1 and its ligand.

102. The method of claim 92, wherein, the immune checkpoint inhibitor is an anti-PD-1 antibody or anti-PD-L1 antibody.

103. The method of claim 92, wherein the immune checkpoint inhibitor is an anti-PD-1 monoclonal antibody, or an anti-PD-L1 monoclonal antibody.

104. The method of claim 92, wherein the immune checkpoint inhibitor is nivolumab, pembrolizumab, cemiplimab, durvalumab, atezolizumab or avelumab.

105. The method of claim 92, wherein the cancer is selected from bladder cancer, colorectal cancer, breast cancer, prostate cancer, renal cancer, hepatocellular cancer, lung cancer, ovarian cancer, urothelial cancer, cervical cancer, gastric cancer, esophageal cancer, head and neck cancer, melanoma, squamous cell carcinoma, Merkel cell carcinoma, mesothelioma, non-small cell lung cancer (NSCLC), small-cell lung cancer, colon cancer; multiple myeloma (MM), Hodgkin's lymphoma (HL), B-cell lymphoma or diffuse large B-cell lymphoma (DLBCL).

106. The method of claim 99, further comprising monitoring the effectiveness of the treatment comprising the step of comparing the level of cells expressing intracellular arginase 1 in a blood sample from the subject after the treatment to the level of cells expressing intracellular arginase 1 before the treatment.
